(19) United States
(12) Patent Application Publication
(10) Pub. No.: US 2019/0001020 A1
(43) Pub. Date: Jan. 3, 2019

(54) COLLAGEN BASED MATERIALS AND METHODS OF USING THEM

(71) Applicants: Hans P.I. Claesson, Lexington, MA (US); Laurence J. Berlowitz, Beverly, MA (US); Matthew Fullana, Wilmington, MA (US); Gary E. Wnek, Cleveland, OH (US)

(72) Inventors: Hans P.I. Claesson, Lexington, MA (US); Laurence J. Berlowitz, Beverly, MA (US); Matthew Fullana, Wilmington, MA (US); Gary E. Wnek, Cleveland, OH (US)

(21) Appl. No.: 15/876,249

(22) Filed: Jan. 22, 2018

Related U.S. Application Data
(63) Continuation of application No. PCT/US16/43113, filed on Jul. 20, 2016.
(60) Provisional application No. 62/195,712, filed on Jul. 22, 2015.
(60) Provisional application No. 62/218,442, filed on Sep. 14, 2015.

(51) Int. Cl. A61L 27/24; A61L 27/36; C08L 89/06; C08J 3/24

(52) U.S. Cl. CPC A61L 27/24; A61L 27/3666; C08J 3/245; C08L 89/06; A61L 27/3687

(57) ABSTRACT

Certain configurations of adhesive materials are described which comprise a crosslinked derivatized atelocollagen. In some configurations, the crosslinked, derivatized atelocollagen is cured to provide a burst strength of at least 55 kPa or 60 kPa (or more) as tested by ASTM F2392-04. In some instances, the crosslinked derivatized atelocollagen comprises a methylated atelocollagen that is crosslinked using one or more functionalized crosslinking agents.

### PRIORITY APPLICATIONS

This application is a continuation application of, and claims priority to and the benefit of, PCT/US16/43113 filed on Jul. 20, 2016. PCT/US16/43113 is related to, and claims priority to and the benefit of, each of U.S. Application No. 62/195,712 filed on Jul. 22, 2015 and U.S. Application No. 62/218,442 filed on Sep. 14, 2015, the entire disclosure of each of which is hereby incorporated herein by reference for all purposes.

### TECHNOLOGICAL FIELD

Certain features, aspects and embodiments are directed to collagen based materials and methods of using them. In particular, certain embodiments are directed to collagen based adhesive/sealant materials with high burst strength that can adhere to biological tissues.

### SUMMARY

Certain features, aspects and embodiments described herein are directed to adhesive or sealant materials comprising atelocollagen that has been derivatized and/or cross-linked to provide the adhesive material.

In one aspect, the derivatized, crosslinked atelocollagen adhesives and/or sealants described herein can be produced such that a user has ample working time to position, spread and/or manipulate the material. In many existing adhesives and sealants (particularly soft tissue sealants), the materials cure almost instantly or in 30 seconds or less, which can create problems if the material is misplaced, too thick, too thin or otherwise need to be manipulated. As noted herein, by selecting suitable conditions to derivatize the atelocollagen, deposit it and cure it, a desired overall procedure time that is not too slow but is not too fast can be achieved.

In some aspects described herein, the collagen used is a high quality collagen, e.g., one that comprises 50% or more by weight of monomeric triple helix collagen. Certain attributes can be achieved (as noted below) by using a high quality collagen and, in particular, a high quality atelocollagen.

In some aspects, an adhesive/sealant material comprising a crosslinked, derivatized atelocollagen that provides a burst strength of at least 55 kPa or at least 60 kPa or more after curing as tested by ASTM F2392-04 is provided.

In other aspects, an adhesive/sealant material comprising a cross-linked, derivatized atelocollagen that has been cross-linked in the presence of at least two different functionalized crosslinking agents where at least one of the two different functionalized crosslinking agents is a functionalized polyethylene glycol crosslinking agent, in which the adhesive material provides a burst strength of at least 55 kPa or at least 60 kPa or more after solid curing as tested by ASTM F2392-04 is described.

In another aspect, an adhesive/sealant material composition comprising a derivatized, atelocollagen present at a concentration of at least 20 mg/mL or at least 30 mg/mL or at least 35 mg/mL, the derivatized, atelocollagen effective to adhere to a biological tissue when crosslinked and cured is disclosed.

In an additional aspect, an adhesive/sealant composition comprising a collagen solution or suspension comprising a derivatized atelocollagen comprising a number average molecular weight of about 300 kDa, a first crosslinking agent, and a second crosslinking agent different from the first crosslinking agent, in which the collagen, the first crosslinking agent and the second crosslinking agent react to provide an adhesive material is provided.

In another aspect, a pre-cured adhesive/sealant composition comprising a mixture of a collagen solution or suspension comprising a derivatized atelocollagen comprising a number average molecular weight of about 300 kDa, a first crosslinking agent, and a second crosslinking agent different from the first crosslinking agent, in which the collagen, the first crosslinking agent and the second crosslinking agent react to provide an adhesive material is described.

In an additional aspect, a pre-cured adhesive composition comprising a mixture of a collagen solution comprising a derivatized atelocollagen with at least about 75% of carboxyl sites of the atelocollagen being derivatized, a first crosslinking agent, and a second crosslinking agent different from the first crosslinking agent, in which the collagen, the first crosslinking agent and the second crosslinking agent react to provide an adhesive material is disclosed.

In another aspect, a pre-cured adhesive composition comprising a mixture of a collagen solution or suspension comprising a derivatized atelocollagen with at least about 75% of carboxyl sites of the atelocollagen being derivatized, in which the derivatized collagen comprises a number average molecular weight of about 300 kDa or less, a first crosslinking agent, and a second crosslinking agent different from the first crosslinking agent, in which the collagen, the first crosslinking agent and the second crosslinking agent react to provide an adhesive material is described.

In another aspect, a kit comprising derivatized atelocollagen, a first crosslinking agent, a second crosslinking agent different from the first crosslinking agent, and instructions for using the derivatized atelocollagen, the first crosslinking agent, and the second crosslinking agent to provide an adhesive material is provided.

In an additional aspect, a kit comprising collagen comprising an atelocollagen, a derivatization agent, a first crosslinking agent, a second crosslinking agent different from the first crosslinking agent, instructions for using the atelocollagen and derivatization agent to provide a derivatized atelocollagen, and instructions for using the provided derivatized atelocollagen, the first crosslinking agent, and the second crosslinking agent to provide an adhesive material is disclosed.

In another aspect, a method of repairing a tissue defect comprising disposing a composition at a defect site of the tissue, the composition comprising a derivatized atelocollagen comprising a number average molecular weight of less than or equal to 300 kDa, a first crosslinking agent and a second crosslinking agent different than the first crosslinking agent, adding a curing agent to the disposed composition, and permitting the curing agent to remain on a surface of the disposed composition for a curing period is provided.

In an additional aspect, a method of repairing an epithelial tissue defect comprising disposing a composition at a defect site of the epithelial tissue, the composition comprising a derivatized atelocollagen comprising a number average molecular weight of less than or equal to 300 kDa, a first crosslinking agent and a second crosslinking agent different than the first crosslinking agent, adding a curing agent to the disposed composition, and permitting the added solid curing agent to remain on a surface of the disposed composition for a curing period.

In an additional aspect, a method of repairing a tissue defect comprising disposing a composition at a defect site of the epithelial tissue, the composition comprising a derivatized atelocollagen, a first crosslinking agent and a second crosslinking agent different than the first crosslinking agent, in which the adhesive material provides a burst strength of at least 60 kPa after curing as tested by ASTM F2392-04, adding a curing agent to the disposed composition; and permitting the curing agent to remain on a surface of the composition for a curing period is provided.

In an additional aspect, a method of repairing an epithelial tissue defect comprising disposing a composition at a defect site of the epithelial tissue, the composition comprising a derivatized atelocollagen, a first crosslinking agent and a second crosslinking agent different than the first crosslinking agent, in which the composition provides a burst strength of at least 60 kPa after curing as tested by ASTM F2392-04, adding a curing agent to the disposed composition, and permitting the added solid curing agent to remain on a surface of the disposed composition for a curing period is disclosed.

In another aspect, a kit is described which comprises collagen comprising an atelocollagen, a derivatization agent, a first crosslinking agent, a second crosslinking agent different from the first crosslinking agent, a solid curing agent comprising an average particle size of less than about 150 microns, instructions for using the atelocollagen and derivatization agent to provide a derivatized atelocollagen, and instructions for using the provided derivatized atelocollagen, the first crosslinking agent, the second crosslinking agent and the curing agent to provide a cured adhesive material.

In an additional aspect, an adhesive/sealant material comprises a cross-linked, derivatized atelocollagen that has been cross-linked in the presence of at least two different functionalized crosslinking agents and has been cured using a solid curing agent comprising an average particle size of less than about 150 microns, the adhesive/sealant material providing a burst strength of about 60 kPa or more after curing as tested by ASTM F2392-04.

In another aspect, a method comprising subjecting dry atelocollagen particles to an acidic solution comprising a derivatizing agent to provide the derivatized collagen, washing the derivatized collagen, and drying the derivatized collagen is provided. In certain instances, the acidic solution comprising the derivatizing agent comprises methanol in hydrochloric acid. In other instances, the atelocollagen particles comprise a mixture of mammalian, predominately Type I, either dermis or tendon-derived ,predominantly monomeric atelocollagen in dry form. In some examples, the washing step comprises washing the derivatized collagen with an alcohol. In other examples, the alcohol is a primary alcohol. In further embodiments, the primary alcohol is selected from the group consisting of methanol, ethanol and propanol. In some instances, the drying step comprises drying using a vacuum. In other examples, the method comprises adding the dried, derivatized collagen to an acidic solution for storage. In some examples, the method comprises the concentration of the derivatized collagen to provide a final concentration of about 30-40 mg/mL of the derivatized collagen. In other embodiments, the method comprises disposing the derivatized collagen onto a tissue site and curing the disposed derivatized collagen. In further examples, the curing step comprises disposing a sodium or calcium salt on the disposed derivatized collagen. In some examples, the sodium or calcium salt is disposed as a solid on the disposed derivatized collagen.

Other aspects and advantages of the present technology will become apparent to those skilled in the art, given the benefit of this summary and the following detailed description.

### BRIEF DESCRIPTION OF THE FIGURES

Certain configurations and illustrative test results are described with reference to the accompanying figures in which

FIG. 1A is an illustration of a NHS-PEG crosslinking agent and FIG. 1B is an illustration of SH-PEG crosslinking agent;

FIG. 2 is a bar graph showing curing times and burst strengths for one illustrative composition, in accordance with certain examples;

FIG. 3 is bar graph comparing liquid curing and solid curing burst strengths, in accordance with certain embodiments;

FIG. 4 is a table listing literature burst strength values for several existing tissue adhesives;

FIG. 5 is an SDS-PAGE gel comparing porcine collagen and bovine collagen, in accordance with certain examples;

FIGS. 6-9 are pictures showing the test results of one material for a pig lung defect, in accordance with certain embodiments;

FIGS. 10-13 are pictures showing the test results of one material for another pig lung defect, in accordance with certain embodiments;

FIGS. 14-17 are pictures comparing a commercially available adhesive (FIGS. 14 and 15) with a test material (FIGS. 16 and 17) in repairing a defect in a pig lung, in accordance with certain examples;

FIG. 18 is a picture showing repair of a defect in a pig aorta, in accordance with certain embodiments;

FIGS. 19 and 20 are pictures showing repair of a defect in a pig colon, in accordance with certain examples;

FIG. 21 is a graph showing comparison of shear rates between porcine atelocollagen and bovine atelocollagen, in accordance with certain examples; and

FIG. 22 is a chart showing burst strengths for different material, in accordance with certain embodiments.

### DETAILED DESCRIPTION

Certain embodiments are described below that are directed to collagen based adhesive/sealant materials that can be used in tissue repair procedures or in the treatment of tissues. While the exact tissue repair method can vary, in a typical procedure the adhesive/sealant material is disposed over the defect or injured area and used to seal the area, e.g., by forming a film or other structure covering the defect. In some instances, the material can be smeared, smoothed, etc. to spread the material as a thin layer over the defect area. Once spread to a desired thickness, the material may then be cured using a suitable curing agent. If desired, the defect area can also be sutured, stapled or tissues can be coupled to each other through other mechanical means, e.g., screws, fasteners, etc. in addition to disposal of the adhesive material at the defect site. While the terms adhesive and sealant are both used, an adhesive may comprise the same or similar materials as a sealant but generally cures in a much longer time, e.g., 10×, 20×, 50×, or 100× or more time, compared to the curing time of a sealant.

While the exact thickness of the cured adhesive material can vary, a typical use thickness may vary from about 0.1 mm to about 2 mm, more particularly about 0.25 mm to about 1.5 mm, for example about 0.5 mm to about 1 mm. In some instances, it may be desirable to apply a thinner layer of material, cure it, and then apply additional material (and cure it) on top of the cured material. The number of layers applied may vary from about two to about ten or more. In certain configurations, layering of the material may be performed after an underlying layer is partially cured but not yet fully cured. For example, where a material cures in about four minutes, a first layer may be applied and then cured for about two minutes followed by application of another layer of the material. In other instances, different materials can be layered or the same material can be layered but different curing agents (or curing agents of the same material but with a different average particle size) can then be added over a cured or partially cured layer.

Collagens and Atelocollagens

In certain embodiments, the compositions described herein may comprise one or more collagens or atelocollagens. An atelocollagen can be produced from a collagen by digesting the collagen with a protease, e.g., pepsin, or other suitable enzymes to remove the atelopeptides from the collagen helix. Without wishing to be bound by any theory, collagen comprise a triple helix including a length of about 300 nm and a weight (in monomeric triple helix form) of about 300 kiloDaltons (kDa). In certain examples, the particular form of collagen selected for use in producing the materials described herein can vary depending on the desired end product and/or the desired types of collagen structures present from different tissues. In some embodiments, collagen obtained from any source, including both naturally occurring collagens and synthetic collagens, can be used to provide the materials described herein. For example, collagen may be extracted and purified from human or other animal sources, e.g., mammalian sources, such as bovine or porcine, or may be recombinantly produced, synthetically produced by chemical synthesis or otherwise produced using selected techniques. Collagen of any type, including, but not limited to, types I-IV or any combination thereof, may be used. In some embodiments, type I collagen is used to provide the materials described herein. In other embodiments, atelopeptide collagen or telopeptide-containing collagen may be used. Atelopeptide collagen, a.k.a., atelocollagen, may include desirable attributes including reduced immunogenicity when compared to telopeptide-containing collagen. In some embodiments, the collagen can be a Type I collagen, a Type II collagen, a Type III collagen, a Type IV collagen, a gelatin, a collagen comprising agarose, a collagen comprising hyaluronan or hyaluronic acid, a collagen comprising proteoglycan, a glycosaminoglycan, a glycoprotein, glucosamine or galactosamine, a collagen comprising fibronectin, a collagen comprising laminin, a collagen comprising a bioactive peptide growth factor, a collagen comprising a cytokine, a collagen comprising elastin, a collagen comprising fibrin, a collagen comprising polylactic, polyglycolic or polyamino acid, a collagen comprising polycaprolactone, collagen comprising a polypeptide, or a copolymer thereof, each alone or in combination. In other examples, the collagen can be prepared from, or can include, collagen precursors, such as, for example, peptide monomers, alpha 1 (type I), and alpha 2 (type I) collagen peptide or alpha 1 (type I) alpha 2 (type I) peptides, alone or in combination, or from a combination of precursors, such as 2 (alpha 1, type I) peptide and 1 (alpha 2, type I) peptide. If desired, the collagens can be used with other compounds, such as pharmaceutically acceptable excipients, surfactants, buffers, additives and other biocompatible components or pharmaceutical agents, therapeutic agents or bioactive agents or molecules.

In some examples, collagen can be isolated from animal tissues, e.g., skin, hides, etc. For example, the tissue can be digested in the presence of one or more enzymes to solubilize the collagen and provide an atelocollagen. Atelocollagens are typically lower in antigenicity than native collagen but can still provide many of the properties of an intact collagen. Atelocollagens can also be soluble (under certain conditions) which can further enhance processing and use. As described herein, however, certain instances can use a solid (and optionally suspended in a solution) atelocollagen which can be derivatized and then reacted with one or more crosslinking agents to provide a sealant, adhesive or similar materials. In some instances, the solid collagen can be reacted with solid (powdered) crosslinking agent and then cured with one or more solid or liquid curing agents. These reactions and curing may take place in one or more vessels, e.g. syringes, tubes, vials, etc. or may take place at a tissue site.

In some embodiments, the atelocollagen can be isolated and stored at temperatures below room temperature, e.g., less than 25° C., 15° C., 10° C., 4° C. or 0° C., to reduce the likelihood of formation of higher ordered structures. For example, collagen and atelocollagens can form triple helix dimers, triple helix trimers, etc. in solution. Formation of these higher ordered structures can reduce the level of monomeric atelocollagen in solution and may provide sealants and adhesives with less desired properties. In certain examples described herein, monomeric triple helix derivatized atelocollagen can be present in a major amount by weight, e.g., more than 50% by weight, in the collagen. In some instances, the collagen may comprise derivatized atelocollagen comprising a number average molecular weight of 300 kDa or less, e.g., a major amount of derivatized atelocollagen comprising a number average molecular weight of 300 kDa or less may be present in the collagen used to provide the sealant, adhesive or other material. Without wishing to be bound by any particular theory, where derivatized atelocollagen comprising a number average molecular weight (NAMW) of 300 kDa or less is used, the overall viscosity (per mg/mL of the material) is reduced compared to collagens used in other collagen based sealants and adhesives. By having a lower viscosity, increased amounts of the derivatized atelocollagen can be present for a particular viscosity, e.g., 40 mg/mL derivatized atelocollagen with a NAMW of 300 kDa can have about the same viscosity as 20 mg/mL of collagen used for prior collagen based adhesives such as those described in U.S. Pat. No. 6,458,889, for example. By having a higher concentration of derivatized atelocollagen present, improved properties can be achieved while maintaining a suitable viscosity for use as an adhesive or sealant.

In certain embodiments, an atelocollagen from porcine sources can be isolated and purified and stored at a temperature of room temperature or below prior to use. The porcine atelocollagen can be derivatized, e.g., alkylated, under suitable conditions, e.g., in the presence of an alkylating agent and/or acid. Alkylation may result in a solid derivatized collagen that can then be used to provide a sealant, adhesive, gel or other similar material. In some instances, the porcine, derivatized atelocollagen can then be reacted with one, two, three or more crosslinking agents (as described below) to provide a sealant, adhesive or other similar material. The porcine, derivatized, cross-linked atelocollagen may then be cured using suitable curing agents, e.g., liquid or solid curing agents, to provide a material with desirable properties.

Derivitization Methods and Reagents

In certain embodiments, the collagen (or atelocollagen) can be derivatized by reacting the collagen with a suitable agent that can form a covalent bond with one or more groups present in the collagen structure. The exact derivatization conditions may vary and may be performed in liquid solution or using gaseous reagents to react with the collagen (or atelocollagen). In some instances, the derivatization process may be performed in a single step in a single reaction vessel, whereas in other instances the derivatization process may be performed in two or more steps and optionally in two or more different vessels.

In some embodiments, the collagen can be derivatized using a suitable alkyl alcohol in the presence of an acid or base (depending on the alcohol used and the desired reaction to be promoted). Illustrative alkyl alcohols include, but are not limited to, methanol, ethanol, propanol, isopropanol, butyl alcohol, isobutyl alcohol, alcohols with alkyl chains having five to eight carbons (saturated or unsaturated), alcohols with alkyl chains having nine to twelve carbons (saturated or unsaturated), alcohols with alkyl chains having thirteen to eighteen carbons (saturated or unsaturated), alcohols with alkyl chains having nineteen to twenty-four carbons (saturated or unsaturated) and substituted forms of these illustrative alcohols. In some embodiments, the derivatizing agent may comprise one or more free hydroxyl groups (—OH) but may not be considered an alcohol, e.g., may be considered a carboxylic acid, an epoxide, an aldehyde or other groups.

In some examples, the collagen or atelocollagen can be derivatized by reacting one or more collagens with a suitable derivatizing agent which may be a pharmaceutically active compound or a pharmaceutically inactive compound. In some embodiments, the derivatizing agent includes at least one carbon atom and a suitable reactive group that can react with one or more groups present in the collagen or atelocollagen. For example, the derivatizing agent can include a hydroxyl group, an amino group or both that can react with a suitable group present on the collagen to couple the derivatizing agent to the collagen. In some embodiments, the derivatizing agent can take the form of an alkylating agent, e.g., an agent that can add a methyl group, ethyl group, propyl group or other alkyl group to the collagen, e.g., C1-C6 alkyl groups that may be saturated or unsaturated or C6-C24 alkyl groups which may be saturated or unsaturated. Alkylated atelocollagens may be particularly desirable due to their potential for improved solubility in a variety of common solvents, the potential for more facile formulation with drugs and various other small-molecule and/or macromolecular additives, and the potential for good mechanical properties resulting from intermolecular interactions between alkyl groups, especially alkyl groups of sufficient length to promote hydrophobic association in aqueous solutions and even crystallization-induced aggregation. Also, the adhesive properties of such modified atelocollagens can be tuned by judicious choice of alkyl-based functional groups, e.g., alkyl groups including long hydrocarbon chains, and spacing of the functional groups along the collagen polymer chain.

In some embodiments, more than a single type of derivatizing agent can be used. For example, collagen or atelocollagen can first be reacted with a first derivatizing agent and then subsequently reacted with a second derivatizing agent. In other embodiments, the same derivatizing agent can be used, but it may be reacted sequentially with the collagen molecule or reacted in selected stoichiometric amounts with the collagen to derivatize a desired proportion of the collagen. In some embodiments, the collagen may first be derivatized with a lower molecular weight alkyl chain (C1-C6) and then subsequently reacted with a higher molecular weight alkyl chain (C10-C18 or C10-C24) to increase the overall hydrophobicity of the derivatized collagen. In other embodiments, the collagen can first be reacted with a lower molecular weight alkyl chain and subsequently reacted with a different group, e.g., a pharmaceutically active compound or a biological agent such as, for example, a peptide, protein, carbohydrate, lipid or other biological agents.

In certain embodiments, the derivatizing agent can be an alkylating agent having one to six carbon atoms including, but not limited to, straight chain, branched and cyclic alkylating agents with one to six carbon atoms. Where 5 or 6 carbon atoms are present, the alkylating agent can be a fully saturated cyclic compound, a cyclic compound with one or more sites of unsaturation, or an aromatic compound. In other embodiments, the alkylating agent can include one carbon, one to two carbons, one to three carbons, one to four carbons, one to five carbons, or one to six carbons. In some examples, the alkylating agent can include two carbons, two to three carbons, two to four carbons, two to five carbons or two to six carbons. In other examples, the alkylating agent can include three carbons, three to four carbons, three to five carbons, or three to six carbons. In certain examples, the alkylating agent can include four carbons, four to five carbons or four to six carbons. In some embodiments, the alkylating agent can include five carbons or five to six carbons. In other embodiments, the alkylating agent can include six carbons. The alkylating agents described herein may be fully saturated or may be present in unsaturated form, e.g., may include one or more double or triple bonds. In some instances herein, alkylating agents including one to six carbon atoms are referred to as “lower weight alkylating agents.”

In certain embodiments, the alkylating agent can include six to ten carbons. Alkylating agents with six to ten carbon atoms are sometimes referred to herein as “middle weight alkylating agents.” In certain examples, the alkylating agent can include six to ten carbons, six to nine carbons, six to eight carbons, or six to seven carbons. In other embodiments, the alkylating agent can include seven to ten carbons, seven to nine carbons or seven to eight carbons. In further embodiments, the alkylating agent can include eight to ten carbons or eight to nine carbons. In other examples, the alkylating agent can include nine carbon atoms or ten carbon atoms. The middle weight alkylating agents described herein may be fully saturated or may be present in unsaturated form, e.g., may include one or more double or triple bonds.

In other embodiments, the alkylating agent can be selected to include more than ten carbon atoms to provide a hydrophobic, derivatized collagen. Alkylating agents including more than ten carbon atoms are referred to herein in certain instances as “high weight alkylating agents.” In some instances, derivatized atelocollagens can be more hydrophobic than native collagen and can be adherent, at least to some degree, to permit use of the derivatized collagen in wound repair, defect repair, skin grafts, as scaffolds to attract cells and permit colonization by cells or other uses. The hydrophobic nature of the derivatized atelocollagen may result in the collagen precipitating out of solution and being considered a solid. The solid can be suspended if desired to permit further reaction. Due to the sticky nature of certain collagens after derivatizing with an alkylating agent, they can also be used as glues or adhesives, or when used as an implant or scaffold can be used without an additional adhesive. While not required, “high weight alkylating agents” may comprise up to eighteen, nineteen, twenty, twenty-one, twenty-two, twenty-three, or twenty-four carbons. In some instances, high weight alkylating agents can take the form of a fatty acid chains such as those found in essential fatty acids and non-essential fatty acids.

In certain examples, where the alkylating agent includes more than ten carbon atoms, it may include ten to twenty four carbons, ten to twenty-two carbons, ten to twenty carbons, ten to eighteen carbons, ten to sixteen carbons, ten to fourteen carbons or ten to twelve carbons and include both saturated and unsaturated forms thereof. In other embodiments, the alkylating agent can include twelve to twenty four carbons, twelve to twenty-two carbons, twelve to twenty carbons, twelve to eighteen carbons, twelve to sixteen carbons, or twelve to fourteen carbons and include both saturated and unsaturated forms thereof. In further embodiments, the alkylating agent can include fourteen to twenty four carbons, fourteen to twenty-two carbons, fourteen to twenty carbons, fourteen to eighteen carbons, or fourteen to sixteen carbons and include both saturated and unsaturated forms thereof. In additional embodiments, the alkylating agent can include sixteen to twenty four carbons, sixteen to twenty-two carbons, sixteen to twenty carbons, or sixteen to eighteen and include both saturated and unsaturated forms thereof. In other embodiments, the alkylating agent can include eighteen to twenty four carbons, eighteen to twenty-two carbons, or eighteen to twenty carbons and include both saturated and unsaturated forms thereof. In some embodiments, the alkylating agent can include twenty to twenty four carbons or, twenty to twenty-two carbons and include both saturated and unsaturated forms thereof. In embodiments where long alkyl chains, e.g., C18 or more, are to be added to the collagen or atelocollagen methods similar to those described by Greenberg and Alfrey in “Side Chain Crystallization of n-Alkyl Polymethacrylates and Polyacrylates,” J. Am. Chem. Soc., 1954, 76 (24), pp. 6280-6285, can be used.

In some embodiments, the alkylating agent comprises at least one terminal reactive group, e.g., a terminal hydroxyl group or terminal amino group. In other embodiments, the alkylating agent comprises at least one internal reactive group, e.g., a hydroxyl group or amino group not present at a terminus of the alkylating agent. In some embodiments, the alkylating agent can be a compound of formula (VI)

R₁—CH₃ (VI)

where R₁ may be hydroxyl, amino, acyl, or benzoyl. In other embodiments, R₁ includes at least one carbon atom, e.g., 1 to 6 carbon atoms, in addition to a non-carbon atom such as, for example, hydrogen, nitrogen or sulfur. In some embodiments, R₁ can be HOCH₂—, HOCH₂CH₂— or HO(CH₂)ₙ— where n is three to ten, more particularly, three to nine, three to eight, three to seven, three to six, three to five, or three, four, five, six, seven, eight, nine or ten. In some embodiments, R₁ of formula (1) is hydroxyl (—OH), a primary amine (—NH₂) or may be a diamine or a sulfhydryl or thiol group (—SH).

In other embodiments, the alkylating agent can be one or more of the compounds of formula (VII)

R₂—CH₂ₙ—CH₃ (VII)

where R₂ may be hydroxyl, amino, acyl or benzoyl and n can be 1, 2, 3, 4, 5, or 6. In some embodiments, n is 1 and R₂ is hydroxyl, e.g., —OH. In other embodiments, n is 2 and R₂ is hydroxyl. In further embodiments, n is 3 and R₂ is hydroxyl. In additional embodiments, n is 4 and R₂ is hydroxyl. In additional embodiments, n is 5 and R₂ is hydroxyl. In some embodiments, n is 1 and R₂ is amino, e.g., —NH₂. In other embodiments, n is 2 and R₂ is amino. In further embodiments, n is 3 and R₂ is amino. In additional embodiments, n is 4 and R₂ is amino. In additional embodiments, n is 5 and R₂ is amino. If desired, where R₂ is amino, the amino group may be a secondary amine or a tertiary amine instead of a primary amine.

In certain embodiments, the alkylating agent can be selected as a compound of (VIII)

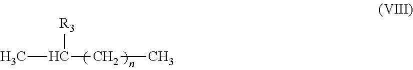

where R₃ may be hydroxyl, amino, acyl or benzoyl and n can be 1, 2, 3, 4, 5 or 6. In some embodiments, n is 1 and R₃ is hydroxyl, e.g., —OH. In other embodiments, n is 2 and R₃ is hydroxyl. In further embodiments, n is 3 and R₃ is hydroxyl. In additional embodiments, n is 4 and R₃ is hydroxyl. In additional embodiments, n is 5 and R₃ is hydroxyl. In some embodiments, n is 1 and R₃ is amino, e.g., —NH₂. In other embodiments, n is 2 and R₃ is amino. In further embodiments, n is 3 and R₃ is amino. In additional embodiments, n is 4 and R₃ is amino. In additional embodiments, n is 5 and R₃ is amino. If desired, where R₂ is amino, the amino group may be a secondary amine or a tertiary amine instead of a primary amine.

In some embodiments, the derivatizing agent can be a compound having formula (IX)

R₂—CH₂ₙ—R₄ (IX)

where R₂ of formula (IX) may be any of those groups listed for R₂ of formula (2) and n is 1, 2, 3, 4, 5 or 6. R₄ can methoxy, ethoxy, oxypropyl, hydroxyl, amino, acyl, benzoyl, aryl, naphthyl or carboxyl (—COOH). In some embodiments, each of R₂ and R₄ is hydroxyl and n is 2, 3, 4, 5, or 6. In other embodiments, one of R₂ and R₄ is hydroxyl and the other of R₂ and R₄ is amino and n is 2, 3, 4, 5 or 6.

In other embodiments, the derivatizing agent can be a compound having formula (X)

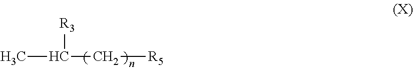

where R₃ of formula (5) may be any of those groups listed for R₃ of formula (3) and n is 1, 2, 3, 4, 5 or 6. R₅ can methoxy, ethoxy, oxypropyl, hydroxyl, amino, acyl, benzoyl, aryl, naphthyl or carboxyl. In some embodiments, each of R₃ and R₅ can be hydroxyl or one of R₃ and R₅ is hydroxyl and the other one of R₃ and R₅ is amino.

In other embodiments, the alkylating agent may be a compound of formula (XI)

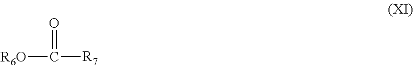

where R₆ can be hydrogen or a hydrocarbon chain (saturated or unsaturated) having 1 to 4 carbon atoms which may be branched if desired. In some embodiments, R₇ comprises 1 to 6 carbons (saturated and unsaturated forms). In other embodiments, R₇ may be a hydrocarbon chain (saturated or unsaturated) including 7-10 carbons. In further embodiments, R₇ may be a hydrocarbon chain (saturated or unsaturated) including ten to twenty four carbon atoms. In some embodiments, R₆ is hydrogen and R₇ is selected to provide compound (6) that is one of dodecanoic acid, tetradecanoic acid, hexadecanoic acid, octadecanoic acid, eicosanoic acid, tetracosanoic acid, 9-hexadecenoic acid, 9-octadecenoic acid, 9,12 octadecadienoic acid, 9,12,15-octadecatrienoic acid, 6,9,12-octadecatrienoic aid, 5,8,11,14-eicosatetraenoic acid or 15-tetracosenoic acid. In some embodiments, R₇ can have a formula of CH₃(CH)ₙ— where n can be nine to about twenty-three. In other embodiments, R₇ can have a formula of CH₃(CH)ₙ—C═O where n can be nine to about twenty-three. In some embodiments, R₆ is hydrogen and R₇ is any one of the groups listed above for R₇.

In other embodiments, the alkylating agent may be a compound of formula (XII)

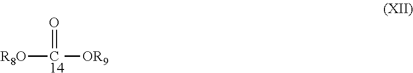

where R₈ can be hydrogen or a hydrocarbon chain (saturated or unsaturated) having 1 to 4 carbon atoms which may be branched if desired, and R₉ may be a hydrocarbon chain (saturated or unsaturated) including ten to twenty four carbon atoms. In some embodiments, R₉ can have a formula of CH₃(CH)ₙ— where n can be nine to about twenty-three. In other embodiments, R₉ can have a formula of CH₃(CH)n-C═O where n can be nine to about twenty-three. In some embodiments, each of R₈ and R₉ is hydrogen. In other embodiments, R₈ is hydrogen and R₉ may be any of the groups listed above for R₉.

In certain embodiments, the atelocollagen may be a methylated atelocollagen comprising one or more methyl groups added to the collagen at a suitable site. For example, the derivatized collagen may be methylated at one or more carboxyl groups to provide an ester. In some embodiments, enough derivatizing agent is present such that substantially all free and accessible carboxyl sites of the collagen react with the derivatizing agent to form an ester comprising a methoxy group covalently bonded to the carbonyl group of the ester.

In other embodiments, the derivatizing agent to provide methylation (or other alkylation or derivatization) can be selected such that free carboxyl groups remain in the collagen molecule subsequent to reaction with the derivatizing agent. By using selected amounts of the derivatizing agent, free reactive groups remain post-derivatization to permit reaction with other agents, e.g., one or more cros slinking agents.

In certain examples, the exact conditions used to derivatize the collagen or atelocollagen may depend on the particular derivatizing agent used. In some embodiments, derivatization may be acid catalyzed or base catalyzed. In additional instances, derivatization can be performed at room temperature, elevated temperature above room temperature or at a temperature below temperature. As noted herein, maintaining the collagen at reduced temperature may reduce the formation of unwanted higher order structures. The form of the derivatizing reagents used may also vary, e.g., gaseous forms and liquid forms can be used.

In some embodiments, the collagen or atelocollagen may be derivatized in the presence of an alkyl alcohol, e.g., methanol, ethanol, propanol, etc. under acidic conditions. For example, gaseous or liquid acid may be present in a reaction vessel with the collagen or atelocollagen, and the alkyl alcohol can be introduced into the reaction vessel to derivatize the collagen or atelocollagen. The exact acid used may depend on the particular alkyl alcohol selected, but illustrative acids include but are not limited to acetic acid, citric acid, perchloric acid, oxalic acid, hydrochloric acid, hypochlorous acid, sulfuric acid, nitric acid, phosphoric acid, hypophosphorous acid, hypophosphoric acid and other acids which may or may not include metal species such as chromium, selenium, manganese or other metals. The pH of any solution used in the derivatization process is desirably not so low that the collagen or atelocollagen is degraded to any substantial degree, and illustrative pH values which can be used vary from about 2 pH units to about 6.5 pH units. If desired, one or more acidic buffers may also be present to keep the pH at a substantially constant pH during the derivatization process.

In certain embodiments, a pharmaceutically active compound can be reacted with the derivatized collagen or atelocollagen to conjugate the active compound to the collagen or atelocollagen. In some instances, the pharmaceutically active compound make take the form of a biological agent or compound, e.g., a peptide, protein, carbohydrate, lipid or the like, that can be conjugated to the derivatized collagens described herein. Illustrations of pharmaceutically active compounds are described in more detail below. In certain examples, a pharmaceutically active compound is a chemical compound, e.g., natural or synthetic, that can elicit a biological response, e.g., activate a cellular response or pathway, inhibit a cellular response or pathway, promote a cellular response or pathway, or alter a cellular response or pathway, under suitable conditions. For example, a pharmaceutically active compound can result in activation (or inactivation) of a cellular pathway, enzyme activation, enzyme inhibition, differentiation of a stem cell into a desired cell type, repair of a tissues, cellular enhancement, inhibition of cellular processes, activation (or deactivation) of an ion channel, an increase (or decrease) in protein expression, an increase (or decrease) in RNA production, an increase (or decrease) in mitosis, an increase (or decrease) in meiosis, an increase (or decrease) in intracellular transport, or other activities that a cell may perform or initiate through one or more cellular systems or pathways.

In certain examples, when a pharmaceutically active compound is present as a conjugate with a derivatized collagen or atelocollagen, e.g., a methylated atelocollagen, it may be present in an effective amount, e.g., an amount effective to elicit the biological response. In some configurations, the concentration of pharmaceutically active compound may exceed the amount desired to elicit a biological response such that sustained release of the pharmaceutically active compound can provide for such biological response for extended periods. It will be recognized by the person of ordinary skill in the art, given the benefit of this disclosure, that there are multiple ways to provide an effective amount of the pharmaceutically active compound. In some embodiments, the pharmaceutically effective amount can be provided by including a plurality of the pharmaceutically active compounds on the derivatized collagen helix, e.g., by including large numbers of conjugated active compound per collagen helix an effective amount can be provided using fewer collagen helices, whereas in other examples the concentration of the derivatized collagen/conjugated compound can be selected to provide the effective amount.

In certain embodiments, the pharmaceutically active compound may be a non-synthetic pharmaceutically active compound whereas in other examples the pharmaceutically active compound may be a synthetic compound. Non-synthetic compounds include, but are not limited to, natural products and naturally occurring compounds. Synthetic compounds include analogs of non-synthetic pharmaceutically active compounds and other compounds not existing naturally or not yet found to exist naturally. Where a non-synthetic pharmaceutically active compound is conjugated to a derivatized collagen, the compound itself may be produced chemically, e.g., using total synthesis, even though the non-synthetic pharmaceutically active compound is naturally occurring. If desired, both a non-synthetic and synthetic pharmaceutically active compound may be conjugated to the derivatized collagen, e.g., to the methylated collagen.

In certain embodiments, the pharmaceutically active compound may be a protein, a carbohydrate, a lipid, a peptide, an amino acid, a nucleoside, a nitrogenous base, a nucleoside phosphate, an interference RNA (RNAi), a steroid, a high energy phosphate, a high energy biomolecule, an enzyme or other compounds, materials or components commonly present in one or more metabolic pathways of a cell.

In certain examples, the derivatized collagens described herein, e.g., methylated atelocollagens, can be conjugated to a growth factor such as, for example, adrenomedullin (AM), autocrine motility factor, a bone morphogenetic protein (BMP), a brain-derived neurotrophic factor (BDNF), an epidermal growth factor (EGF), erythropoietin (EPO), a fibroblast growth factor (FGF), a glial cell line-derived neurotrophic factor (GDNF), a granulocyte colony-stimulating factor (G-CSF), a granulocyte macrophage colony-stimulating factor (GM-CSF), a growth differentiation factor-9 (GDF9), a hepatocyte growth factor (HGF), a hepatoma-derived growth factor (HDGF), an insulin-like growth factor (IGF), a migration-stimulating factor, myostatin (GDF-8), a nerve growth factor (NGF) and other neurotrophins, a platelet-derived growth factor (PDGF), thrombopoietin (TPO), a transforming growth factor alpha(TGF-α), a transforming growth factor beta(TGF-β), a tumor necrosis factor-alpha (TNF-α), a vascular endothelial growth factor (VEGF), a Wnt Signaling Pathway protein, a placental growth factor (PlGF), a fetal bovine somatotrophin (FBS), IL-1-Cofactor for IL-3 and IL-6, IL-2-T-cell growth factor, IL-3, IL-4, IL-5, IL-6, IL-7 or other suitable growth factors. In some examples, any growth factor which can activate (or inhibit if desired) a kinase pathway, e.g., MAP kinase, PI3 kinase or the like, can be conjugated to the derivatized collagen. While the exact chemistry used to couple the growth factor to the derivatized collagen can vary, in many instances a free amino group of the growth factor can be coupled to a free carboxyl group of the collagen to provide an amide bond between the derivatized collagen and the growth factor. Where no such groups are present on the growth factor, the growth factor can be derivatized using one or more activating agents, e.g., a carbodiimide, a succinyl group, etc., and then reacted with a derivatized collagen.

In other configurations, the derivatized collagen can be conjugated to one or more therapeutics such as those described in Goodman and Gilman's the Pharmacological Basis of Therapeutics. Where a therapeutic is present, the therapeutic may be used alone as a pharmaceutically active compound or with another pharmaceutically active compound such as, for example, a growth factor or other selected active compound. Illustrative therapeutic agents include but are not limited to a neurotransmitter, a cholinase activator or inhibitor, an acetylcholinesterase activator or inhibitor, an acetylcholine, an acetylcholine agonist or derivative, a nicotinic receptor agonist, a nicotinic receptor antagonist, a muscarinic receptor agonist, a muscarinic receptor antagonist, dopamine, a dopamine derivative, a catecholamine, an adrenergic receptor agonist, an adrenergic receptor antagonist, an anticholinesterase agent, a nicotinic cholinergic receptor agonist, a nicotinic cholinergic receptor antagonist, a ganglionic blocking compound, a ganglionic stimulant, a serotonin receptor agonist, a serotonin receptor antagonist, an ion channel agonist, an ion channel antagonist, a neuromodulator, a therapeutic gas (e.g., oxygen, carbon dioxide, nitric oxide), ethanol or an ethanol derivative, a benzodiazepine analog, a phenothiazine, a thioxanthene, a heterocyclic compound, a sedative, a norepinephrine reuptake inhibitor, an antidepressant, a serotonin reuptake inhibitor, a monoamine oxidase agonist, a monoamine oxidase antagonist, a sodium ion channel activator, a sodium ion channel inhibitor, a calcium ion channel activator, a calcium ion channel inhibitor, a hydantoin, a barbituate, a stilbene, an iminostilbene, a succinimide, an oxazolidinedione, an antiseizure agent, an analgesic, an opioid, a peptide, an opioid agonist, an opioid antagonist, an autocoid, histamine, a histamine analog, a H1-receptor agonist, a H1-receptor antagonist, a H3-receptor agonist, a H3-receptor antagonist, an eicosanoid, a prostaglandin, a leukotriene, an anti-inflammatory agent, an antipyretic, a nonsteroidal anti-inflammatory agent, a salicylic acid derivative, a salicylate, a para-aminophenol derivative, an indole, an indene, an indole acetic acid, an indene acetic acid, a heteroaryl acetic acid, a propionic acid, an arylpropionic acid, an anthranilic acid, an enolic acid, an alkanone, an oxicam, gold and gold derivatives, a uricosuric agent, a corticosteroid, a bronchodilator, a diuretic, a vasopressin receptor agonist, a vasopressin receptor antagonist, angiotensin, an angiotensin analog, renin, a renin analog, an inhibitor of the renin-angiotensin system, an angiotensin receptor agonist, an angiotensin receptor antagonist, a renin inhibitor, an endopeptidase inhibitor, an organic nitrate, a calcium channel blocker, a beta-adrenergic receptor antagonist, an alpha-adrenergic receptor antagonist, an antiplatelet agent, an antithrombotic agent, an antihypertensive, a benzothiadiazine, a sympatholytic agent, a vasodilator, an angiotensin converting enzyme inhibitor, an angiotensin II receptor antagonist, a cardiac glycoside, a dopaminergic receptor agonist, a phosphodiesterase inhibitor, an antiarrhythmic drug, an HMG CoA reductase inhibitor, an H2 histamine receptor antagonist, an antibiotic, a hydrogen-potassium ATPase inhibitor, an antacid, a laxative, an antidiarrheal agent, an antiemetic agent, a prokinetic agent, oxytocin, an antimalarial agent, a diaminopyrimidine, quinine and quinine derivatives, quinoline and quinoline derivatives, an antihelminthic agent, an antimicrobial agent, a sulfonamide, a quinolone, a penicillin, a cephalosporin, a beta-lactam, an aminoglycoside, a tetracycline, a chloramphenicol, an erythromycin, an isonicotinic acid compound and derivatives thereof, a macrolide, a sulfone, an antifungal agent, an imidozole, a triazole, an antiviral agent, a protease inhibitor, an antiretroviral agent, a reverse transcriptase inhibitor, an acyclic nucleoside phosphonate, a nitrogen mustard, an ethylenimine, a methylmelamine, an alkyl sulfonate, a nitrosourea, a triazene, a folic acid analog, a pyrimidine analog, a purine analog, a vinca alkaloid, an epipodophyllotoxin, a coordination complex, a platinum coordination complex, an anthracenedione, a substituted urea, a methylhydrazine derivative, an adrenocortical suppressant, a progestin, an estrogen, an anti-estrogen, an androgen, an anti-androgen, a gonadotropin-releasing hormone analog, an immunosuppressant, an interferon, a granulocyte macrophage-colony stimulating factor, a tumor necrosis factor, an interleukin, an antibody, an antigen, a hematopoietic agent, an anticoagulant, a hormone, a growth hormone, a glucocorticoid, an antiseptic, insulin, a hypoglycemic agent, a hyperglycemic agent, an insulin analog, a vitamin, a water soluble vitamin, a fat soluble vitamin, a skin agent, an ocular agent, a cosmetic agent, a heavy metal antagonist, or other suitable synthetic or non-synthetic therapeutics. Where a therapeutic agent is present, it may be present alone or in combination with a biomolecule or another pharmaceutically active compound.

In some embodiments, the derivatized collagen described herein can include more than a single type of pharmaceutically active compound conjugated to it. For example, the derivatized collagen can first be reacted with a growth factor and subsequently reacted with a different pharmaceutically active compound to provide a hybrid derivatized collagen comprising more than a single type of pharmaceutically active compound.

In some embodiments, some portion of the derivatized collagen can be derivatized with a suitable active compound and the remainder of the derivatized collagen may not include any derivatized compound. For example, to control the particular level of one or more agents in a sealant or adhesive composition, a derivatized atelocollagen conjugated to a pharmaceutically active compound can be mixed with a derivatized atelocollagen lacking any pharmaceutically active compound. The two collagens or atelocollagens can be mixed or can be layered, e.g., the collagen comprising the active compound may be placed adjacent to the tissues or cells and then covered by the collagen lacking any active compound. Where layers of derivatized collagens are added, each layer can be cured before subsequent layers are added or the layers can first be built up with curing agent added only to the outer layer of the layered construct.

In certain instances, solid collagen material which has been dried and generally free from any solvent or water can be added to a solution comprising a suitable agent to derivatize the collagen. For example, dry solid collagen pieces, particles, powders, etc. can be added to a solvent mixture comprising a derivatizing agent optionally in the presence of an acid. The solid collagen may be dried using many different techniques including but not limited to, lyophilization, blotting, vacuum drying and other techniques which can remove any liquid from the surfaces and bulk of the collagen. If desired, the solid collagen material can be washed with a volatile solvent, e.g., acetone or diethyl ether, to remove any water from the surface of the material.

In some examples, the solvent mixture to which the solid collagen is added may comprise an acidic solution comprising one or more agents which can react with the solid collagen. For example, a strong acid with a low boiling point, e.g., hydrochloric acid, prepared with methanol as the solvent reactant (instead of using water to dilute the acid using aqueous dilute acid) can be used to derivatize, e.g., methylate, the solid collagen. The exact nature of the acid can vary and in certain instances, the acid may be hydrochloric acid, sulfuric acid, nitric acid, phosphoric acid, or any other strong acid. It may also be possible to use weaker acids such as, for example, acetic acid, but the overall acid concentration when a weak acid is present would typically be higher than when a strong acid is used.

In other instances, the solvent reactant mixture may comprise one or more non-aqueous solvents, e.g., organic solvents, including, but not limited to, acetone, diethyl ether, butane, pentane, hexane, benzene or other solvents. For example, the solvent reactant mixture may comprise both an organic solvent an acid in methanol (or other alcohol). In some instances, the solvent mixture may comprise a combination of acetone and acid in methanol (or other alcohol). In other instances, the solvent mixture may comprise diethyl ether and acid in methanol (or other alcohol). In further instances, the solvent mixture may comprise acetone, diethyl ether and an acid in methanol (or other alcohol). Other combinations of organic solvent and acid in methanol (or other alcohol) can also be used.

Crosslinking Agents and Conditions

In some examples, the crosslinking agents used herein may comprise one or more groups which can react with one or more amino acids present in the collagen or atelocollagen. For example, the collagen or atelocollagen typically comprises free amine groups (which may be N-terminal or present in one or more side chains), free carboxyl groups (which may be C-terminal or present in one or more side chains), free sulfhydryl groups (which are typically present in one or more side chains) or other reactive groups which can be derivatized. In some embodiments, two or more different crosslinking agents can be used so different groups present in the collagen or atelocollagen can be derivatized or one crosslinking agent may first react with the collagen or atelocollagen to form a first derivatized collagen or atelocollagen which then reacts with the second cross-linking agent to form a further derivatized collagen or atelocollagen.

In some embodiments, the cross-linking agent may comprise one or more groups including but not limited to, succinimidyl groups, hydroxysuccinimidyl groups, N-hydroxysuccinimidyl groups, sulfhydryl groups, amino groups and other suitable groups.

In some embodiments where two or more crosslinking agents are used one of the crosslinking agents may comprise one or more electrophilic groups and a second crosslinking agent may comprise one or more nucleophilic groups.

In some embodiments, the crosslinking agent(s) may be a synthetic polymer which includes one or more nucleophilic or electrophilic groups. For example, one or both of the crosslinking agents may comprise a synthetic polypeptide or an alkylene glycol, e.g., ethylene glycol, propylene glycol, polyethylene glycol, polypropylene glycol or other polyether based compounds, comprising one or more electrophilic groups or one or more nucleophilic groups. In some embodiments, one of the crosslinking agents used is a polyalkylene glycol comprising one or more electrophilic groups and the other crosslinking agent used is a polyalkylene glycol comprising one or more nucleophilic groups.

In some embodiments, one of the crosslinking agents may comprise a structure of formula (I)

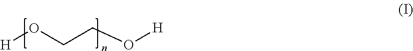

where n may vary between one to sixteen, for example, one to twelve, two to twelve, three to twelve, four to twelve, five to twelve, six to twelve, seven to twelve, eight to twelve, one to eight, two to eight, three to eight, four to eight, five to eight, six to eight, seven to eight, one to six, two to six, three to six, four to six, five to six, one to four, two to four, three to four, or one, two, three, four, five, six, seven, eight, nine, ten, eleven, twelve or other values less than sixteen. If desired, however, the number of chains comprising formula (I) can be greater than sixteen, e.g., up to 100 or more. In some embodiments, formula I may be present in a polyethylene glycol compound which is branched, star shaped, or has a comb geometry.

In other instances, compound comprising one or more of formulae (II)-(IV) may be used as a crosslinking agent.

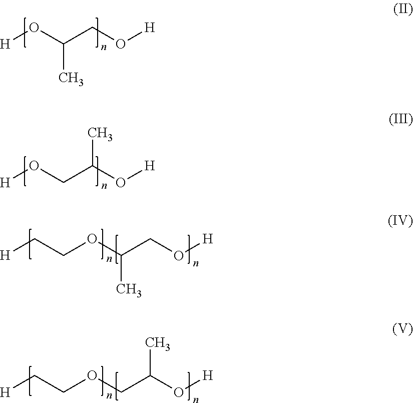

In any of formulae (II)-(V), n may be the same as the values described in connection with formula (I), e.g., two, three, four, five, six or more.

In certain embodiments, a compound including any of formula (I)-(V) may comprise at least one electrophilic group or at least one nucleophilic group. For example, one or more of formula (I)-(V) may comprise an electrophilic group(s) or a nucleophilic group(s). In some instances, a polyethylene glycol may be used that comprises one, two, three, four, five, six or more electrophilic groups or one, two, three, four, five, six or more nucleophilic groups. In some embodiments, each “arm” of the polyethylene glycol compound may comprise a respective nucleophilic or electrophilic group or at least one arm of a multi-arm polyethylene glycol compound may comprise a nucleophilic or electrophilic group. In certain instances, the polyethylene glycol may be a 2-armed PEG, a 4-armed PEG, an 8-armed PEG, a 12-armed PEG, a 16-armed PEG any one of which may comprise at least one nucleophilic or electrophilic group. In some embodiments, the groups present within a single PEG may be the same or may be different, e.g., different nucleophilic groups (or electrophilic groups) can be present on different arms of a PEG or each arm of a PEG may comprise the same nucleophilic groups (or electrophilic groups).

In some examples, the exact type and nature of the nucleophilic groups present in the crosslinking agent can vary and include, but are not limited to, —NH₂, —SH, —OH, —CO—NH—NH₂, etc. The number of nucleophilic groups present can vary from one to more than sixteen, e.g., one, two, three, four, five, six, seven, eight, nine, ten, eleven, twelve or more and may be the same or different. Similarly, the exact type and nature of the electrophilic groups present in the crosslinking agent can vary and include, but are not limited to succinimidyl, hydroxysuccinimidyl, N-hydroxysuccinimidyl, —CO₂—N(COCH₂)₂, —CO₂H, —CHO, —CHOCH₂, —N═C═O, —SO₂—CH═CH₂, —N(COCH)₂, —S—S—(C₅H₄N), etc., and may be the same or different. In certain embodiments, PEGs may be chemically modified to comprise primary amino or thiol groups according to known methods, e.g., Chapter 22 of Poly(ethylene Glycol) Chemistry: Biotechnical and Biomedical Applications, J. Milton Harris, ed., Plenum Press, N.Y. (1992). Various forms of modified PEGs are also commercially available from numerous sources.

In certain embodiments, multi-electrophilic polymers for use in with the collagens described herein may comprise two or more succinimidyl groups capable of forming covalent bonds with nucleophilic groups of the other crosslinking agent or the collagen. For example, succinimidyl groups can be highly reactive with materials containing primary amino (—NH₂) groups, such as multi-amino PEG, poly(lysine), or collagen. Succinimidyl groups are slightly less reactive with materials containing thiol (—SH) groups, such as multi-thiol PEG or synthetic polypeptides containing multiple cysteine residues, though the exact reactivity often depends on the particular reaction conditions including, for example, pH and temperature.

In some embodiments, one of the crosslinking agents described herein comprises at least one succinimidyl group and the other crosslinking agents comprises at least one amino group (primary or secondary) or at least one thiol (—SH) group. In other instances, one of the crosslinking agents comprises a two arm PEG comprising two succinimidyl groups and the other crosslinking agent comprises a two arm PEG comprising two of at least one amino group (primary or secondary) or at least one thiol (—SH) group, e.g., may comprise two amino groups, one amino group and one thiol group or two thiol groups. In other embodiments, one of the crosslinking agents comprises a four arm PEG comprising at least two succinimidyl groups, e.g., four succinimidyl groups, and the other crosslinking agent comprises a four arm PEG comprising two of at least one amino group (primary or secondary) or at least one thiol (—SH) group, e.g., may comprise two or more amino groups, at least one amino group and at least one thiol group or two or more thiol groups. Where the crosslinking agents each comprise a PEG, the PEGs may be the same armed PEGs, e.g., two 4-armed PEGs can be used, or may have different number of arms, e.g., one PEG may be a 4-armed PEG and the other PEG may be a 2-armed PEG or an 8-armed PEG.

In some embodiments, one or more crosslinking agents as described in any of U.S. Pat. Nos. 5,784,500, 6,312,725, 5,162,430, 5,151,689 or related continuation or continuation-in-part applications may be used. In some instances, at least two crosslinking agents from the aforementioned patents can be used with the derivatized collagens and in the processes described herein.

Curing Agents and Conditions

In certain embodiments, once the derivatized collagen is crosslinked, the material can be placed at a desired site and cured using liquid or solid curing agents. Where liquid curing agents are used, the liquid curing agents typically comprise one or more dissolved salts or species that can assist in “hardening” of the gel and increase the overall strength of the material.

In some embodiments, the curing agent composition does not include any aldehyde compounds, e.g., glutaraldehyde, as such compounds can often result in killing of cells. In particular, glutaraldehyde free compositions comprising cured, derivatized and cross-linked atelocollagen materials are particularly desirable for use in applications that contact living cells or tissues. As noted herein, crosslinking of the collagen can result in eventual formation of a gel which may be deposited onto a desired tissue site. The gel form of the material may not provide suitable adhesive properties to retain two tissues in place. In some instances, addition of a curing agent to the disposed collagen material can cure or harden the material and increase its physical properties, e.g., increase its burst strength.

Illustrative curing agents include but are not limited to sodium salts (e.g., sodium chloride, sodium nitrate, sodium phosphate, sodium carbonate, sodium acetate, sodium citrate, sodium bicarbonate, sodium bisulfate, sodium perchlorate, sodium gluconate, sodium lactate), potassium salts (e.g., potassium chloride, potassium nitrate, potassium phosphate, potassium carbonate, potassium acetate, potassium citrate, potassium bicarbonate, potassium bisulfate, potassium perchlorate, potassium gluconate, potassium lactate), calcium salts (e.g., calcium chloride, calcium carbonate, calcium phosphate, calcium acetate, calcium citrate, calcium gluconate, calcium lactate) and mixtures thereof. Any one or more of the salts can be dissolved in a buffer or solvent at a desired pH, e.g., a pH of less than 7, a pH of about 7 to about 7.5 or a pH greater than 7.5. For example, where a basic curing agent solution is desired, a pH of about 8-8.5 may be used. Where an acidic curing agent solution is desired, a pH of about 6-6.5 may be used. In some instances, the one or more salts can be added directly to water without any pH adjustment. Once the salts are dissolved, the curing agent solution may be sprayed onto the disposed, crosslinked, derivatized collagen to cure or harden the collagen.

In some instances, the average particle size of the salts used can be selected to enhance curing and/or burst strength of the cured material. For example and without wishing to be bound by any particular theory, by using one or more salts with an average particle size below about 150 microns or below about 125 microns, the final burst strength of the material may be greater than when the same salt is used at an average particle size above about 150 microns. In certain configurations, a single salt with an average particle size of less than about 150 microns may be used as the curing agent.

In other instances, the salt or salts used may comprise divalent cations. For example calcium or magnesium salts can be used, e.g., calcium or magnesium salts with an average particle size of 150 microns or less can be used either alone or together. In other instances, the divalent cation may be a divalent transition metal, e.g., copper, silver, cobalt, etc. The exact anion used with the divalent cation salt may vary and includes, but is not limited to, halides, carbonates, nitrates, sulfates and other anions. In some examples, the anion may be selected such that the divalent cation salt is not completely water soluble.

In some embodiments, desired physical properties of the collagen material can be achieved by using solid or powder curing agents, e.g., curing agents lacking any liquid and/not dissolved in any solvent. Strength may be enhanced by using solid or powder curing agents which can act to dehydrate the gel and increase the overall collagen weight percentage (and reduce the percentage by weight of water) of the resulting deposited material. In some embodiments, the curing agent can be sprayed on using an air brush or other desired devices. In certain instances, a first curing agent is deposited or blown onto the surface of a deposited collagen material, and then an optional second curing agent may be deposited or blown onto the first curing agent. This two-step curing may improve properties relative to a single step curing. In other instances, however, two or more curing agents may first be mixed and then added to blown onto the deposited collagen gel to permit curing to occur. Where solid curing agents are used, the agents may be any of those curing agents described herein in connection with agents that can be dissolved or are used in a liquid form. In some instances, a first curing agent present as a liquid can be deposited on the collagen, and a second curing agent present as a solid can be added or blown into the first curing agent. If desired, a first curing agent present as a solid can be added or blown onto the deposited collagen, and a second curing agent present as a liquid can deposited on the first curing agent.

In certain embodiments, excess curing agent may be added to ensure full curing takes place in a desired time, e.g., 4 minutes or less, 5 minutes or less, 6 minutes or less, 7 minutes or less, 8 minutes or less, 9 minutes or less or 10 minutes or less. If desired, any excess curing agent can be removed by wiping, washing or otherwise adding some material to the surface of the deposited collagen to remove any residual curing agent. In other instances, excess curing agent may be permitted to remain in place without any washing steps or wiping steps.

In certain embodiments, a film of curing agent may be placed over the disposed, crosslinked collagen or atelocollagen. For example, a film may be cast from a solution comprising one or more curing agents optionally in combination with one or more carriers such as, for example, a polysaccharide, a glucan, a dextran or other suitable carriers. In use, the solid film of curing agent may be disposed onto an area comprising spread crosslinked, derivatized collagen or atelocollagen and may remain in contact with the crosslinked, derivatized collagen or atelocollagen for a curing period. In some instances, the curing agent film can then be removed, whereas in other instances, the curing agent film may remain in place. Illustrative materials which can be used to provide a curing agent film include, but are not limited to, sodium salts (e.g., sodium chloride, sodium nitrate, sodium phosphate, sodium carbonate, sodium acetate, sodium citrate, sodium bicarbonate, sodium bisulfate, sodium perchlorate, sodium gluconate, sodium lactate), potassium salts (e.g., potassium chloride, potassium nitrate, potassium phosphate, potassium carbonate, potassium acetate, potassium citrate, potassium bicarbonate, potassium bisulfate, potassium perchlorate, potassium gluconate, potassium lactate), calcium salts (e.g., calcium chloride, calcium carbonate, calcium phosphate, calcium acetate, calcium citrate, calcium gluconate, calcium lactate) and mixtures thereof. In one illustration, a curing agent film may comprise about 0.10-0.20 M sodium carbonate, 0.10-0.20 M monosodium phosphate and 3% by weight dextran.

In some embodiments materials other than dextrans to form a film curing agent. For example, where the film is used to spread the derivatized, crosslinked atelocollagen, the film may comprise sufficient materials to permit spreading without substantial tearing of the film. The film may also permit the salts present in the film to dissolve on contact with the derivatized, crosslinked atelocollagen. In some instances, one or more plasticizers can be added to the film to alter the properties of the curing agent film. For example, for every 4 mL of a 2.5 weight percent dextran solution comprising 0.15 M monosodium phosphate and 0.15 M sodium carbonate, about one to two drops of glycerin can be added. The resulting material (about 0.5-1 mL) can be deposited onto a support, e.g., a polyethylene sheet, and permitted to dry and form a film. The film may be disc shaped or can be trimmed to a similar shape as the spread derivatized, crosslinked atelocollagen so it can be placed over the derivatized, crosslinked atelocollagen. The disc may be left in place or may be removed after a certain period.

In certain examples, one or more additional stimuli such as cooling, light, chemical initiators or other physical or chemical stimulus can also be added to assist in curing of the derivatized, crosslinked atelocollagen. For example, the salt solutions used in certain curing agents may act to cure the derivatized, crosslinked atelocollagen from the outside in with the outer portion curing faster than a portion adjacent to the tissue. To assist in curing underlying material of the derivatized, crosslinked atelocollagen, one or more stimuli may be applied.

In certain instances, the curing film itself may be used to spread the crosslinked, derivatized collagen or atelocollagen at a defect site. For example, in use of the crosslinked, derivatized collagen or atelocollagen, the material is typically dispensed from a syringe over a tissue defect site. The material may then be spread to a thin film of a desired thickness, e.g., about 0.1 mm to about 1.5 mm, using a suitable tool such as a thin rigid piece of plastic or other materials. If desired, the curing agent film can be cast onto the surface of the plastic such that spreading of the crosslinked, derivatized collagen or atelocollagen results in contact of the crosslinked, derivatized collagen or atelocollagen with the curing agent film. In other instances, the curing agent film itself may be rigid enough to spread the material over the defect site and at the same time cure the crosslinked, derivatized collagen or atelocollagen as the curing agent film spreader contacts the crosslinked, derivatized collagen or atelocollagen.

In certain embodiments, additional materials, additives, etc. may also be used with the collagen and/or curing agent. For example, isoprene based materials may be added to the derivatized collagen to alter the flexibility of the cured material. In other instances, a biological material such as, for example, elastin may be included to mimic tissue flexibility. In further instances, powders, whiskers, fibers, particles, nanoparticles or other materials may also be included in the curing agent or mixed with the derivatized collagen material.

Uses and Applications

The materials produced from the derivatized collagen, the crosslinking agents and the cuing agents can be used as a tissue sealant or adhesive. As noted herein, the presence of atelocollagen can provide materials which are non-immunogenic or have reduced immunogenicity compared to native collagens.

In certain embodiments, the materials described herein may be used to seal or repair incisions or defects in epithelial tissues to each other for at least some period. For example, the material can be used to seal defects, incisions, cuts or openings in skin, esophagus, lung tissue, ear tissues such as the tympanic membrane, the gastrointestinal tract (e.g., stomach, small intestine, large intestine, etc.), the reproductive tract, the urinary tract, exocrine gland tissue, endocrine gland tissue, the surface of the cornea, blood vessels, lymphatic vessels, the pericardium, the pleurae and the peritoneum and other epithelial tissues. As noted in the specific examples below, the materials are particularly effective at sealing defects in pulmonary tissues, blood vessels such as veins and arteries and the gastrointestinal tract. In the case of pulmonary tissue, the materials can repair defects which are air tight under normal physiological pressure to permit proper functioning of the lungs. In the case of blood vessels, the material can repair defects which are liquid tight to permit proper circulatory pressure in veins or arteries. In the case of the gastrointestinal tract, the materials can repair defects to seal the intestinal lining to prevent intestinal material escaping into the abdomen.

In other instances, the materials described herein can be used to seal or repair incisions or defects in muscle tissue including smooth muscle, skeletal muscle and cardiac muscle. To effectuate a repair, it may be desirable to isolate or immobilize the muscle tissue subsequent to curing of the defect with the materials described herein. For example, the materials described herein can be flexible and can stretch to some degree, but excessive movement of the muscle fibers could lead to failure of the cured material. If desired, to increase the overall flexibility and/or strength of the material elastomers, fibers or other materials can be mixed with the derivatized collagen materials prior to curing to alter the properties in a desired manner.

In additional examples, the materials described herein can be used to repair defects in connective tissue including, for example, membranes, nerve coverings, meninges such as spinal cord meninges, connective tissues in the lymphatic system, white adipose tissue, brown adipose tissue, cartilage, bone and other types of connective tissues. If desired or needed, reinforcing materials such as fibers may be mixed with the derivatized collagen materials prior to curing to alter the properties in a desired manner.

In some examples, the materials described herein can be used as a wound sealant or burn sealant to seal defects or injuries in the skin. If desired, the materials can be mixed with stem cells to repair a wound, burn or other tissue defect. In additional instances, the derivatized collagen materials can be used in combination with a plurality of bone precursor cells, a plurality of cartilage precursor cells, or a plurality of epithelial cells. For example, the derivatized collagen materials can be mixed with precursor cells capable for forming into pancreatic cells, e.g., islet cells and/or Beta cells, to assist in the treatment of diabetes or other pancreatic conditions such as cancer. Similarly, the derivatized collagen materials can be used as a loading agent to load precursor cells of any mammalian organ and permit placement and retention of those cells at a desired site. In some instances, the precursor cells may be present in a layer or film below a cured layer or film of the derivatized collagen materials to permit the precursor cells to migrate into an area of the tissue. Growth factors, differentiation factors and the like may also be present with the precursor cells to assist the precursor cells in promoting tissue growth and/or in directing the precursor cells to differentiate into one or more desired cell or tissue types.

In some embodiments, the derivatized collagen or derivatized atelocollagen can be used to deliver one or more pharmaceutically active compounds. For example, hydrophobic pharmaceutically active compounds can be solubilized in hydrophobic domains of derivatized collagen with the collagen acting as reservoirs for such compounds. Where the derivatized collagen is used as a delivery agent, the pharmaceutically active compound is not covalently bound to the derivatized collagen but is instead suspended or solubilized in the derivatized collagen. In some instances, the pharmaceutically active compound make take the form of a biological agent or compound, e.g., a peptide, protein, carbohydrate, lipid or the like, that can be conjugated to the derivatized collagens described herein. Illustrations of pharmaceutically active compounds are described in more detail below. In certain examples, a pharmaceutically active compound that can be delivered using the derivatized collagen can be a chemical compound, e.g., natural or synthetic, that can elicit a biological response, e.g., activate a cellular response or pathway, inhibit a cellular response or pathway, promote a cellular response or pathway, or alter a cellular response or pathway, under suitable conditions. For example, a pharmaceutically active compound can result in activation (or inactivation) of a cellular pathway, enzyme activation, enzyme inhibition, differentiation of a stem cell into a desired cell type, repair of a tissues, cellular enhancement, inhibition of cellular processes, activation (or deactivation) of an ion channel, an increase (or decrease) in protein expression, an increase (or decrease) in RNA production, an increase (or decrease) in mitosis, an increase (or decrease) in meiosis, an increase (or decrease) in intracellular transport, or other activities that a cell may perform or initiate through one or more cellular systems or pathways.

In certain examples, when a pharmaceutically active compound is present in a mixture with a derivatized collagen or atelocollagen, e.g., a methylated atelocollagen, it may be present in an effective amount, e.g., an amount effective to elicit the biological response. In some configurations, the concentration of pharmaceutically active compound may exceed the amount desired to elicit a biological response such that sustained release of the pharmaceutically active compound can provide for such biological response for extended periods. It will be recognized by the person of ordinary skill in the art, given the benefit of this disclosure, that there are multiple ways to provide an effective amount of the pharmaceutically active compound.

In certain embodiments, the pharmaceutically active compound to be delivered may be a non-synthetic pharmaceutically active compound whereas in other examples the pharmaceutically active compound to be delivered may be a synthetic compound. Non-synthetic compounds include, but are not limited to, natural products and naturally occurring compounds. Synthetic compounds include analogs of non-synthetic pharmaceutically active compounds and other compounds not existing naturally or not yet found to exist naturally. Where a non-synthetic pharmaceutically active compound is delivered using a derivatized collagen, the compound itself may be produced chemically, e.g., using total synthesis, even though the non-synthetic pharmaceutically active compound is naturally occurring. If desired, both a non-synthetic and synthetic pharmaceutically active compound may be delivered using a derivatized collagen, e.g., to the methylated collagen.

In certain embodiments, the pharmaceutically active compound that can be delivered using a derivatized collagen may be a protein, a carbohydrate, a lipid, a peptide, an amino acid, a nucleoside, a nitrogenous base, a nucleoside phosphate, an interference RNA (RNAi), a steroid, a high energy phosphate, a high energy biomolecule, an enzyme or other compounds, materials or components commonly present in one or more metabolic pathways of a cell.

In certain examples, the derivatized collagens described herein, e.g., methylated atelocollagens, can be used to deliver a growth factor such as, for example, adrenomedullin (AM), autocrine motility factor, a bone morphogenetic protein (BMP), a brain-derived neurotrophic factor (BDNF), an epidermal growth factor (EGF), erythropoietin (EPO), a fibroblast growth factor (FGF), a glial cell line-derived neurotrophic factor (GDNF), a granulocyte colony-stimulating factor (G-CSF), a granulocyte macrophage colony-stimulating factor (GM-CSF), a growth differentiation factor-9 (GDF9), a hepatocyte growth factor (HGF), a hepatoma-derived growth factor (HDGF), an insulin-like growth factor (IGF), a migration-stimulating factor, myostatin (GDF-8), a nerve growth factor (NGF) and other neurotrophins, a platelet-derived growth factor (PDGF), thrombopoietin (TPO), a transforming growth factor alpha(TGF-α), a transforming growth factor beta(TGF-β), a tumor necrosis factor-alpha (TNF-α), a vascular endothelial growth factor (VEGF), a Wnt Signaling Pathway protein, a placental growth factor (PlGF), a fetal bovine somatotrophin (FBS), IL-1-Cofactor for IL-3 and IL-6, IL-2-T-cell growth factor, IL-3, IL-4, IL-5, IL-6, IL-7 or other suitable growth factors. In some examples, any growth factor which can activate (or inhibit if desired) a kinase pathway, e.g., MAP kinase, PI3 kinase or the like, can be delivered using a derivatized collagen.

In other configurations, the derivatized collagen can be used to deliver one or more therapeutics such as those described in Goodman and Gilman's the Pharmacological Basis of Therapeutics. Where a therapeutic is present, the therapeutic may be used alone as a pharmaceutically active compound or with another pharmaceutically active compound such as, for example, a growth factor or other selected active compound. Illustrative therapeutic agents include but are not limited to a neurotransmitter, a cholinase activator or inhibitor, an acetylcholinesterase activator or inhibitor, an acetylcholine, an acetylcholine agonist or derivative, a nicotinic receptor agonist, a nicotinic receptor antagonist, a muscarinic receptor agonist, a muscarinic receptor antagonist, dopamine, a dopamine derivative, a catecholamine, an adrenergic receptor agonist, an adrenergic receptor antagonist, an anticholinesterase agent, a nicotinic cholinergic receptor agonist, a nicotinic cholinergic receptor antagonist, a ganglionic blocking compound, a ganglionic stimulant, a serotonin receptor agonist, a serotonin receptor antagonist, an ion channel agonist, an ion channel antagonist, a neuromodulator, a therapeutic gas (e.g., oxygen, carbon dioxide, nitric oxide), ethanol or an ethanol derivative, a benzodiazepine analog, a phenothiazine, a thioxanthene, a heterocyclic compound, a sedative, a norepinephrine reuptake inhibitor, an antidepressant, a serotonin reuptake inhibitor, a monoamine oxidase agonist, a monoamine oxidase antagonist, a sodium ion channel activator, a sodium ion channel inhibitor, a calcium ion channel activator, a calcium ion channel inhibitor, a hydantoin, a barbituate, a stilbene, an iminostilbene, a succinimide, an oxazolidinedione, an antiseizure agent, an analgesic, an opioid, a peptide, an opioid agonist, an opioid antagonist, an autocoid, histamine, a histamine analog, a H1-receptor agonist, a H1-receptor antagonist, a H3-receptor agonist, a H3-receptor antagonist, an eicosanoid, a prostaglandin, a leukotriene, an anti-inflammatory agent, an antipyretic, a nonsteroidal anti-inflammatory agent, a salicylic acid derivative, a salicylate, a para-aminophenol derivative, an indole, an indene, an indole acetic acid, an indene acetic acid, a heteroaryl acetic acid, a propionic acid, an arylpropionic acid, an anthranilic acid, an enolic acid, an alkanone, an oxicam, gold and gold derivatives, a uricosuric agent, a corticosteroid, a bronchodilator, a diuretic, a vasopressin receptor agonist, a vasopressin receptor antagonist, angiotensin, an angiotensin analog, renin, a renin analog, an inhibitor of the renin-angiotensin system, an angiotensin receptor agonist, an angiotensin receptor antagonist, a renin inhibitor, an endopeptidase inhibitor, an organic nitrate, a calcium channel blocker, a beta-adrenergic receptor antagonist, an alpha-adrenergic receptor antagonist, an antiplatelet agent, an antithrombotic agent, an antihypertensive, a benzothiadiazine, a sympatholytic agent, a vasodilator, an angiotensin converting enzyme inhibitor, an angiotensin II receptor antagonist, a cardiac glycoside, a dopaminergic receptor agonist, a phosphodiesterase inhibitor, an antiarrhythmic drug, an HMG CoA reductase inhibitor, an H2 histamine receptor antagonist, an antibiotic, a hydrogen-potassium ATPase inhibitor, an antacid, a laxative, an antidiarrheal agent, an antiemetic agent, a prokinetic agent, oxytocin, an antimalarial agent, a diaminopyrimidine, quinine and quinine derivatives, quinoline and quinoline derivatives, an antihelminthic agent, an antimicrobial agent, a sulfonamide, a quinolone, a penicillin, a cephalosporin, a beta-lactam, an aminoglycoside, a tetracycline, a chloramphenicol, an erythromycin, an isonicotinic acid compound and derivatives thereof, a macrolide, a sulfone, an antifungal agent, an imidozole, a triazole, an antiviral agent, a protease inhibitor, an antiretroviral agent, a reverse transcriptase inhibitor, an acyclic nucleoside phosphonate, a nitrogen mustard, an ethylenimine, a methylmelamine, an alkyl sulfonate, a nitrosourea, a triazene, a folic acid analog, a pyrimidine analog, a purine analog, a vinca alkaloid, an epipodophyllotoxin, a coordination complex, a platinum coordination complex, an anthracenedione, a substituted urea, a methylhydrazine derivative, an adrenocortical suppressant, a progestin, an estrogen, an anti-estrogen, an androgen, an anti-androgen, a gonadotropin-releasing hormone analog, an immunosuppressant, an interferon, a granulocyte macrophage-colony stimulating factor, a tumor necrosis factor, an interleukin, an antibody, an antigen, a hematopoietic agent, an anticoagulant, a hormone, a growth hormone, a glucocorticoid, an antiseptic, insulin, a hypoglycemic agent, a hyperglycemic agent, an insulin analog, a vitamin, a water soluble vitamin, a fat soluble vitamin, a skin agent, an ocular agent, a cosmetic agent, a heavy metal antagonist, or other suitable synthetic or non-synthetic therapeutics. Where a therapeutic agent is present, it may be present alone or in combination with a biomolecule or another pharmaceutically active compound.

In some embodiments, the derivatized collagen described herein can be used to deliver more than a single type of pharmaceutically active compound. For example, the derivatized collagen can be mixed with a growth factor and a different pharmaceutically active compound to provide a three component mixture.

In certain embodiments, the materials described herein can be mixed with one or more pharmaceutically active agents or biologically active agents and used, at least in part, to deliver these agents to a specific tissue site. For example, crosslinked, derivatized atelocollagen can be mixed with one or more pharmaceutically active agents or biologically active agents prior to curing. The resulting combination may be disposed at a desired site to permit the agent to be in proximity to the site. The material may then be cured as described herein. In some embodiments where the collagen is mixed with a pharmaceutically active agent or biologically active agent, it may be desirable to layer the material to provide some uncured or partially cured material closest to the tissue site and a cured covering or cured cover layer to seal the pharmaceutically active agents or biologically active agents underneath. By keeping the layer adjacent to the tissue at least partially fluid, the pharmaceutically active agents or biologically active agents can diffuse into the tissue and provide some therapeutic effect.

Certain specific test results are described below to illustrate further some of the conditions and uses of the materials described herein.

### EXAMPLE 1

Solid atelocollagen obtained from porcine hide (from Nippon Ham, Japan) was methylated using the following two-step process: (1) the solid atelocollagen was placed in a solution of methanol/hydrochloric acid (a solution prepared in methanol containing 0.9 M diethyl ether and 0.1 M hydrochloric acid) at room temperature for 48 hours, and (2) the resulting product was washed 3× with ethanol. After washing, the methylated atelocollagen was vacuum dried for 18 hours. No further washing steps or any washing steps with an aqueous solution were performed prior to use.

The resulting methylated atelocollagen was about 90% methylated (as verified using a colorimetric spectroscopic method using a hydroxylamine compound and the number of carboxyl groups calculated present in the atelocollagen) and could be loaded at a level of about 38 mg/mL (as determined by BCA analysis) to provide a desired viscosity. The number average molecular weight of the methylated collagen was 300 kDa or less. A higher quality collagen (one containing higher amounts of monomeric collagen vs dimer/trimeric) has a lower viscosity for a selected concentration. This permits loading of the collagen at a higher level (e.g., 38 mg/mL or more) to provide a desired viscosity.

A native PAGE gel comparing the methylated atelocollagen with non-methylated atelocollagen shows that the methylated atelocollagen migrated further than the non-methylated atelocollagen. This result is consistent with the methyl group forming an ester with carboxylic acid groups and removing the negative charge from those groups, which provides a more overall positive charge for the methylated atelocollagen molecules.

### EXAMPLE 2

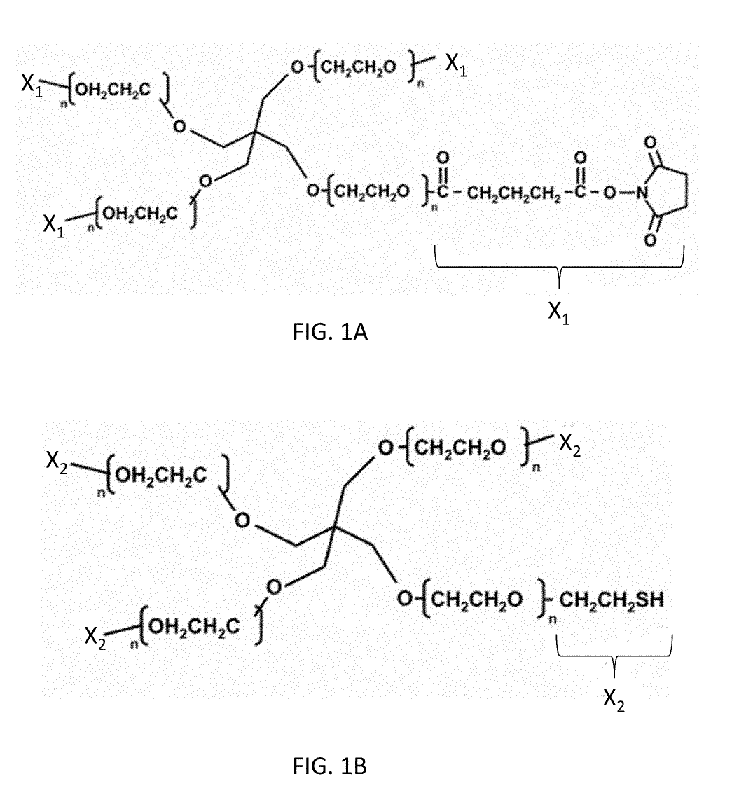

The methylated atelocollagen produced in Example 1 was crosslinked in the presence of two crosslinking agents. The methylated atelocollagen was present along with a solution at a pH of about 3.5-4.1 in a first syringe. The two crosslinking agents (one 4-arm NHS-PEG (see FIG. 1A) and one 4-arm SH-PEG (see FIG. 1B)) purchased commercially from NOF America Corp. (White Plains, N.Y.)) were placed together as powders in a second syringe. The acidic methylated atelocollagen was injected from the first syringe into the second syringe to mix the components. Any air bubbles were removed/minimized from the syringe.

### EXAMPLE 3

The resulting crosslinked, methylated atelocollagen was applied to a surgery site and spread to a thin film (about 1 mm thickness). Either a liquid curing component (sodium carbonate/monosodium phosphate in a 0.3 M aqueous solution at a pH of 9.7) or a solid curing component (about 30 mg of 1:1 mixture by weight of solid sodium carbonate/solid sodium monophosphate) was added to the thin film of atelocollagen to cure the collagen. Any excess curing agent was removed by rinsing using saline or Ringer's solution. In the case of solid curing agent, any excess was also removed by wiping.

### EXAMPLE 4

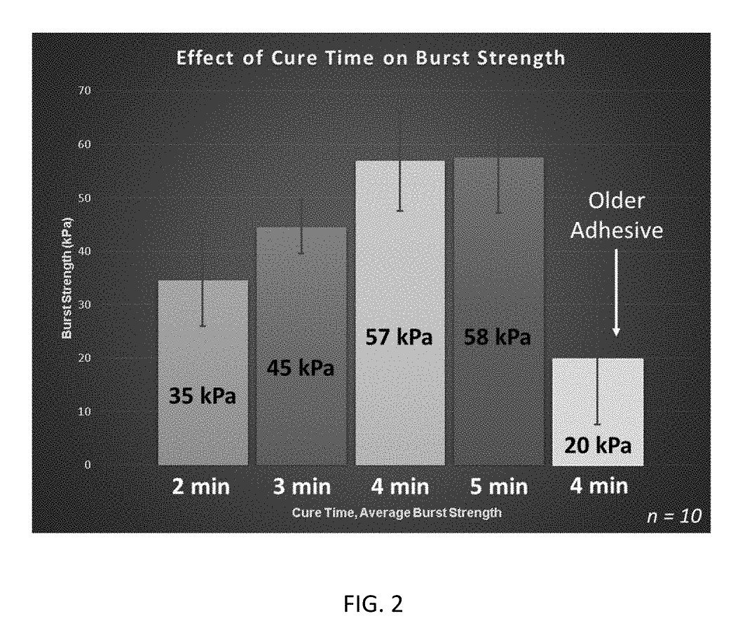

Referring to FIG. 2, a graph showing burst strength measurements of the 1 mm film from Example 3 is shown. Burst strength measurements were measured according to ASTM F2392-04 entitled “Standard Test Method for Burst Strength of Surgical Sealants” dated 2004. In brief, a sausage casing was punctured using a 3 mm surgical punch. A 15 mm in diameter and 1 mm thick layer of the material from Example 3 was placed over the punched perforation and allowed to cure for a minimum of 2 minutes after application of a liquid curing agent referenced in Example 3. The sealed casing is subjected to increasing pressure until rupture. The maximum pressure read on a pressure gauge is recorded as the burst strength.

Referring to FIG. 2, the material showed a burst strength that increased up to about 58 kPa at a cure time of 4 minutes. At two minutes, the material appears to not be fully cured, but curing appeared to be complete at 4 minutes. In comparison, many existing adhesive materials have a burst strength of 20 kPa or less (see far right column in FIG. 2).

### EXAMPLE 5

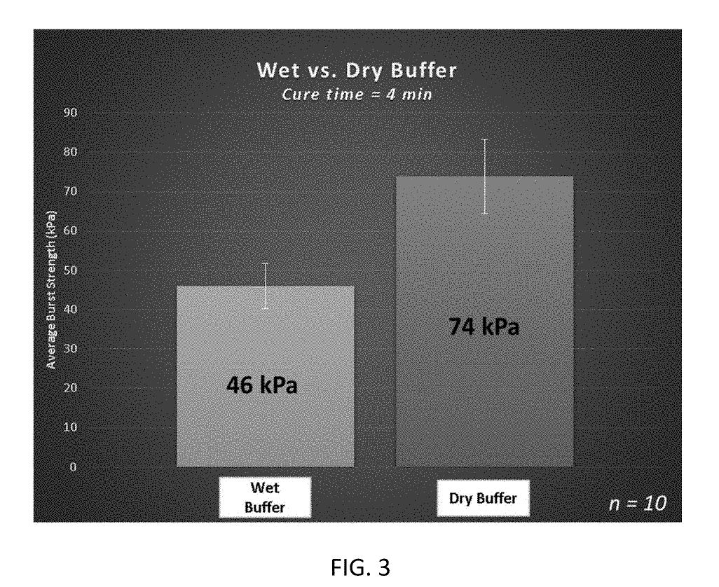

To determine the effect of liquid versus solid curing agent on the material, both the liquid curing agent from Example 3 and the solid curing agent from Example 3 were compared using a 0.5 mm film of the crosslinked, methylated atelocollagen. Referring to FIG. 3, the results were consistent with the solid curing agent providing a higher burst strength (74 kPa) versus a liquid curing agent (46 kPa). About a 60% improvement in burst strength was observed using dry or solid curing as compared to liquid curing. Further, in comparing the burst strength of the 1 mm film of Example 4 to the 0.5 mm film of Example 5, the solid cured thinner film of Example 5 had a higher burst strength even though it was 50% thinner than the liquid cured film used in Example 4.

### EXAMPLE 6

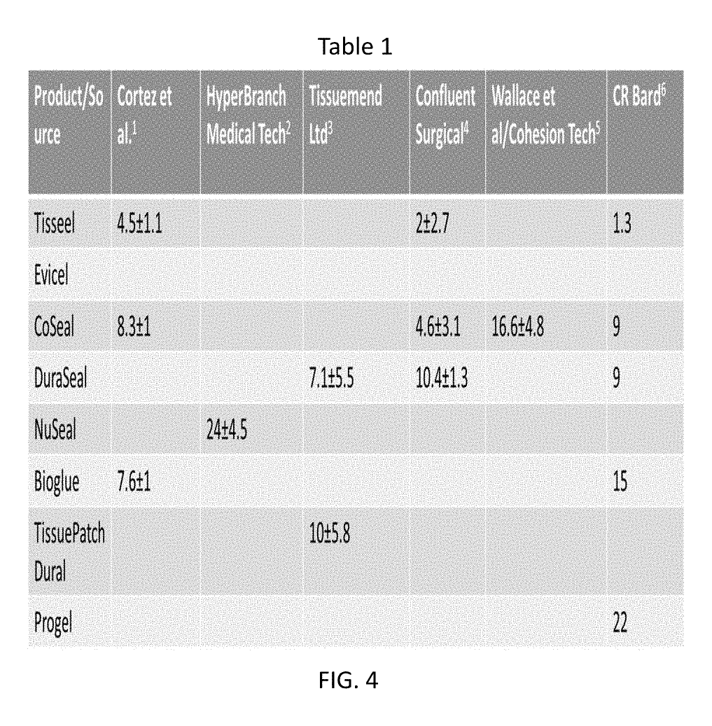

Table 1 in FIG. 4 shows a literature comparison of several adhesives to each other. As shows in the table, none of the existing adhesives had a burst strength of over about 22 kDa. In comparison, adhesives produced using the materials described herein can provide burst strengths of 2×, 3× or more than those listed in Table 1.

### EXAMPLE 7

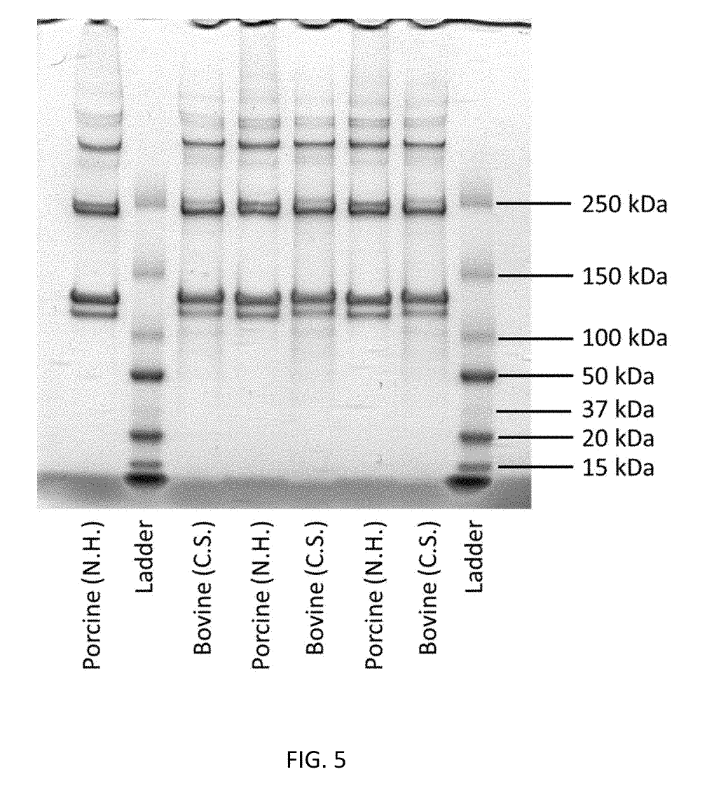

An SDS-PAGE gel analysis was performed to compare porcine and bovine collagens (see FIG. 5). The porcine collagen was the same collagen used in Example 1, and the bovine collagen was obtained from Collagen Solutions. The bovine collagen showed a greater fraction of lower molecular weight components, which are representative of impurities. The porcine collagen was much cleaner and included fewer impurities compared to the bovine collagen.

### EXAMPLE 8

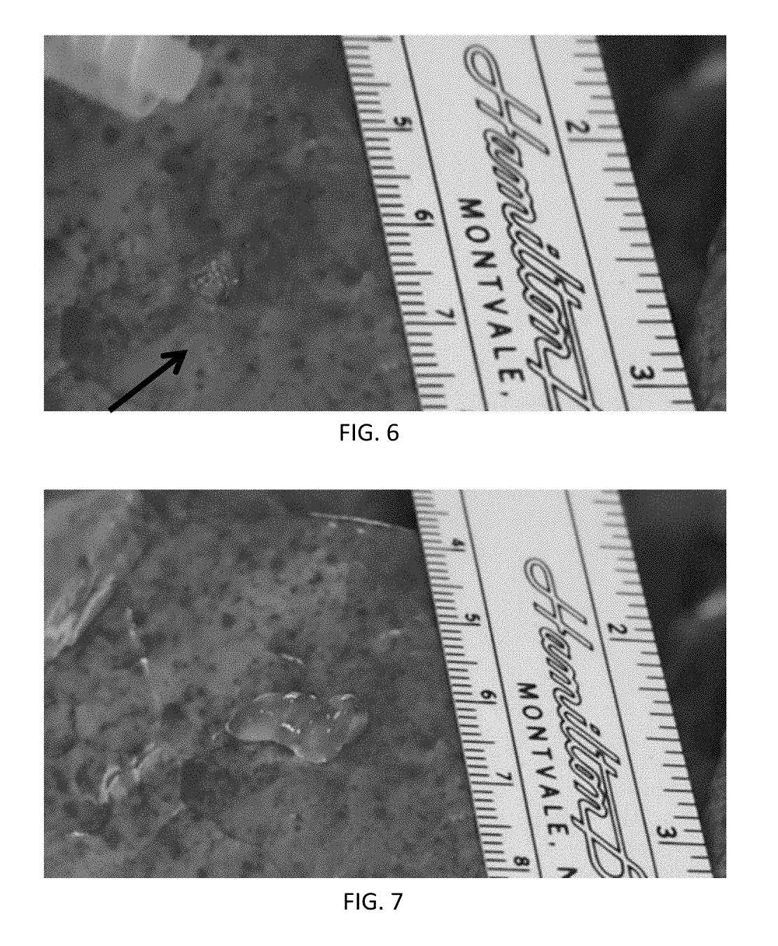

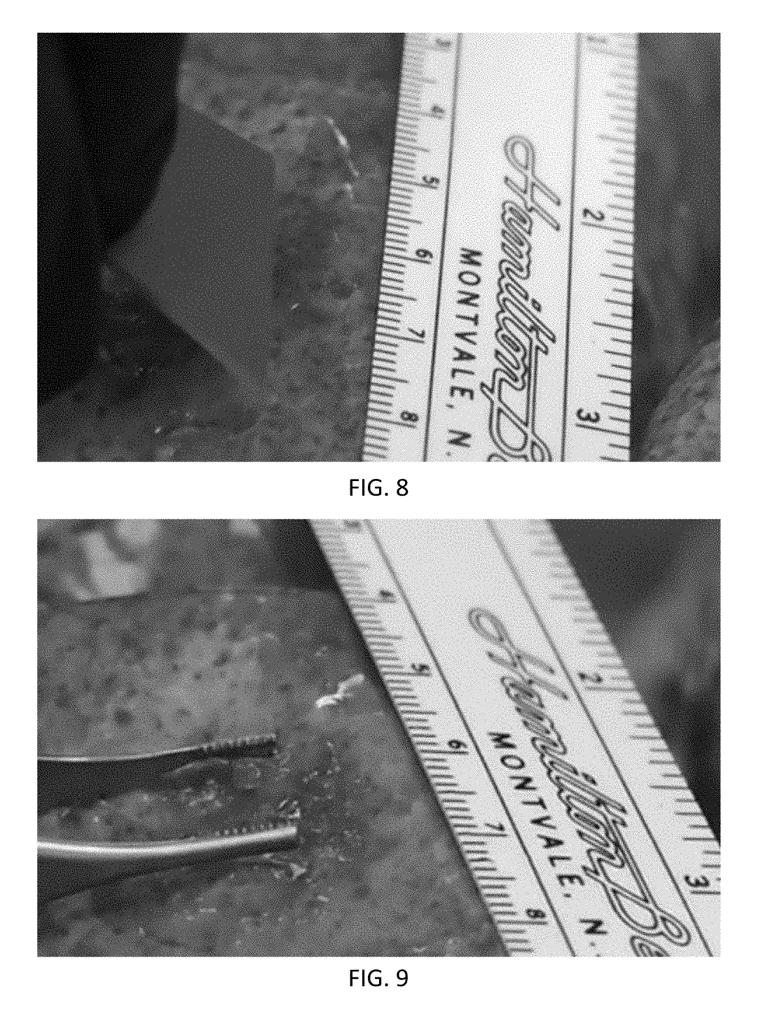

To further test the material produced in Examples 1-3, the material was applied to ex vivo pig lung. The material was applied over a 0.5 cm circular defect in the right cranial lobe of the pig lung (see arrow in FIG. 6). The material was dispensed from a syringe to cover the defect (see FIG. 7). The material was smoothed over the defect using a flat piece of plastic (see FIG. 8). Liquid curing agent from Example 3 was added to cure the material. After five minutes, the material appeared as a film that could not be removed by scraping the defect area with forceps (see FIG. 9).

The lung was placed under water and air pressure was applied (about 40 mm of Hg to inflate the lung) to determine if any liquid or gas could penetrate through the defect. No leaks through the sealed defect site were observed.

### EXAMPLE 9

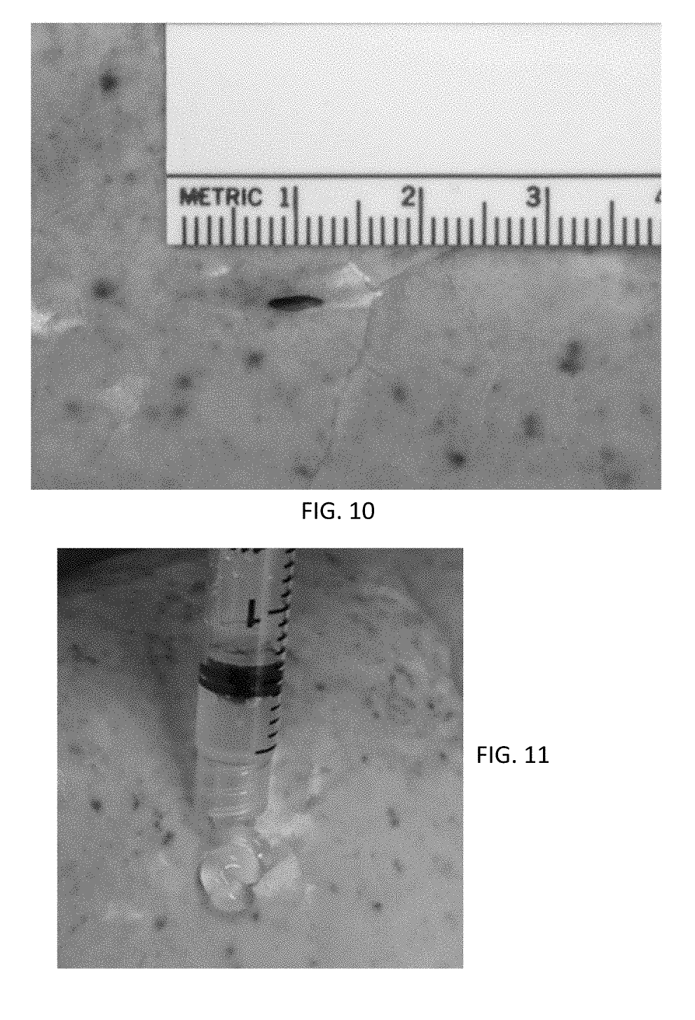

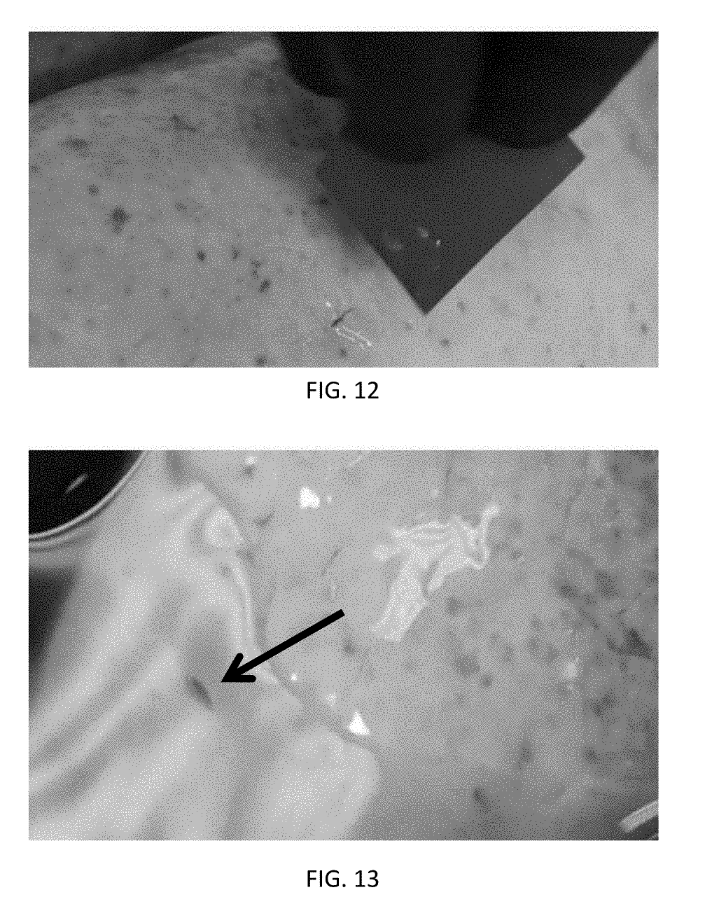

A 0.5 cm linear defect was created in the left caudal lobe of a pig lung (see FIG. 10). The material was dispensed from a syringe to cover the defect (see FIG. 11). The material was smoothed over the defect using a flat piece of plastic (see FIG. 12). Liquid curing agent from Example 3 was squirted on top of the deposited material. After 5 minutes of curing, the defect area was submersed in water and air pressure was applied (about 40 mm of Hg to inflate the lung) to determine if any liquid or gas could penetrate through the defect (see FIG. 13). No leaks through the sealed defect site (see arrow in FIG. 13) were observed.

### EXAMPLE 10

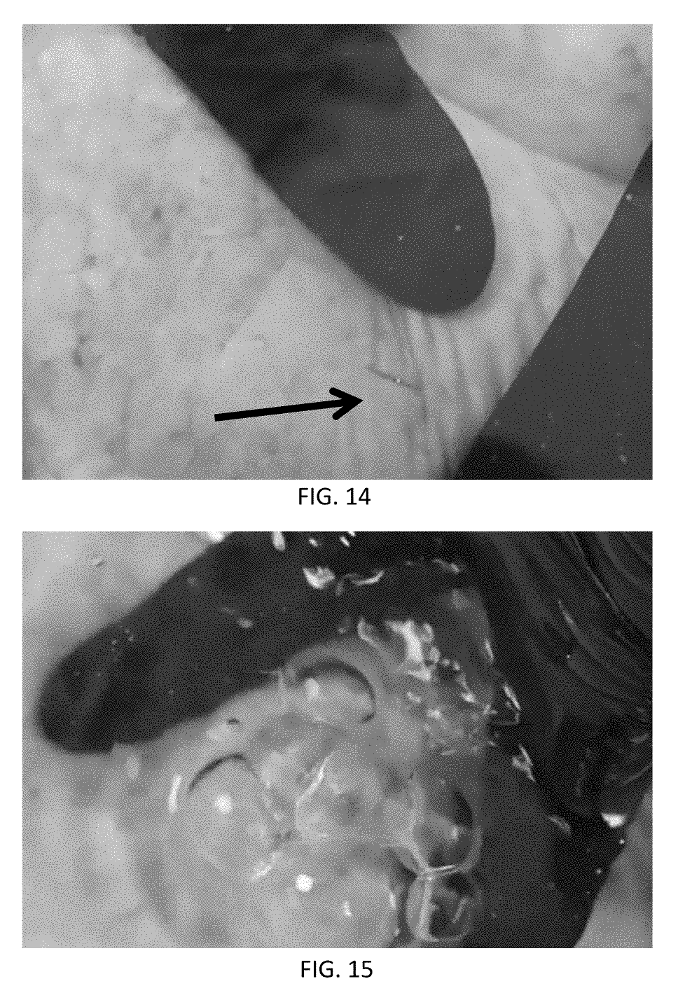

Further tests were performed on the material using 1 cm linear defect in a pig lung. The material of Examples 1-3 was compared to a Progel™ material (commercially available from BARD (Warwick, R.I.)), which is a polyethylene glycol (PEG) and human serum albumin (HSA) based material. The Progel™ material was first applied to the linear defect after wiping the defect surface and drying it. The Progel™ material was dispensed from syringes onto the defect according to its use instructions. After a 2 minute cure time, the area was rinsed with saline. The repair appeared to be intact and was smooth to the touch (see arrow in FIG. 14). When submersed in water without any air pressure, the sealed area appeared to adhere well to the lung surface. The lung was then inflated to about 20 mm Hg pressure and air bubbles immediately escaped from the sealed defect site (see FIG. 15).

A second application of the Progel™ material was then applied over the first. After the second application, the defect again appeared to be sealed. After submerging the lung in water and application of air pressure to inflate the lung (about 20 mm Hg pressure), bubbles were again observed leaking from the defect.

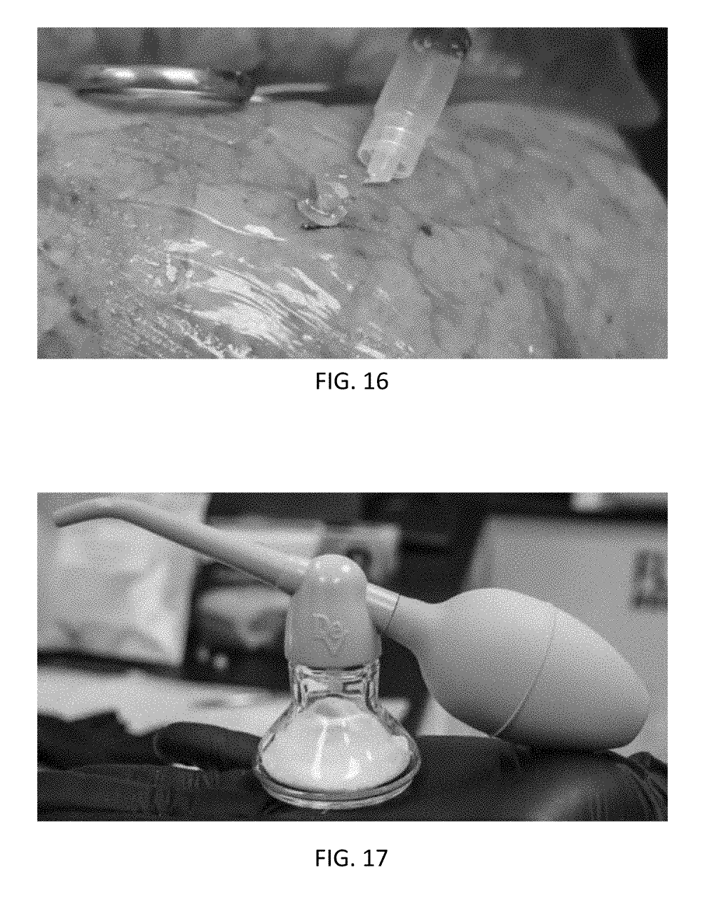

The material from Examples 1-3 was then applied over the defect site (see FIG. 16) and smoothed as before. A duster (see FIG. 17) including powdered sodium carbonate/monosodium phosphate was used to dust the surface of the lung containing the smoothed material and cure the material. The solid curing agent was allowed to remain on the surface for about 4 minutes. The site was then flushed with saline to remove any curing agent. The lung was then submersed in water and inflated to a pressure of about 20 mm Hg. No air bubbles or leakage was observed post inflation.

### EXAMPLE 11

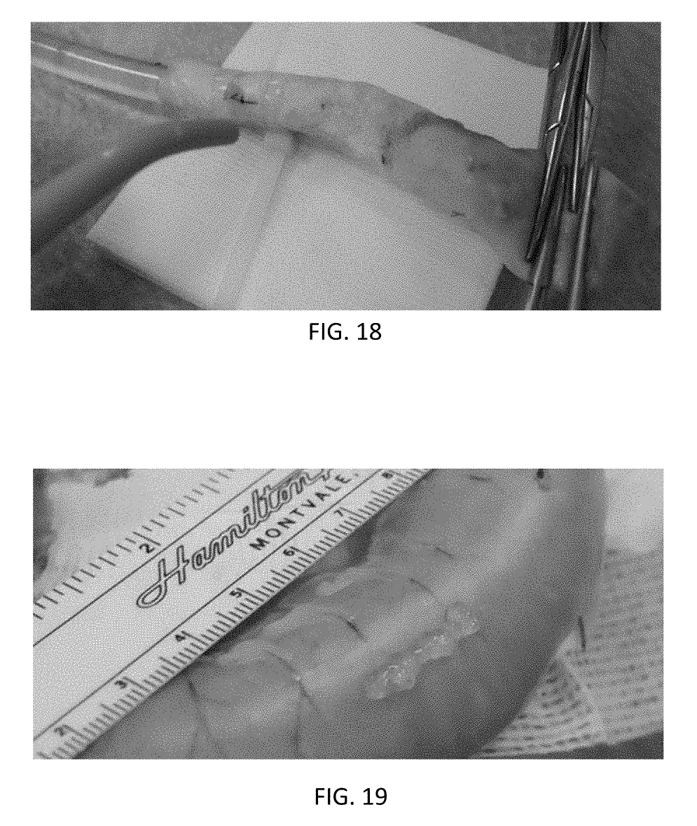

To test the material further, a pig aorta was clamped at one end and liquid pressure was applied from a tube introduced into the aorta at the other end. 1 cm linear incision was made in the aorta. Leakage from saline at the incision site of fluid introduced through the tube was observed. The material of Examples 1-3 was added to the incision site and smoothed over as before. Solid curing agent was applied in a similar manner as in Example 10 (see FIG. 18). The solid curing agent remained on the surface for about 4 minutes, and then the surface was flushed with saline to remove any solid material. The repair was leak tested using saline and an applied pressure of 100 mm Hg. No leaks were observed at the repaired incision site, however leaks began to occur at areas remote from the repaired incision site. At 100 mm Hg pressure, the system leaked at the tube/aorta interface, but no leaks were observed at the repaired tissue site.

### EXAMPLE 12

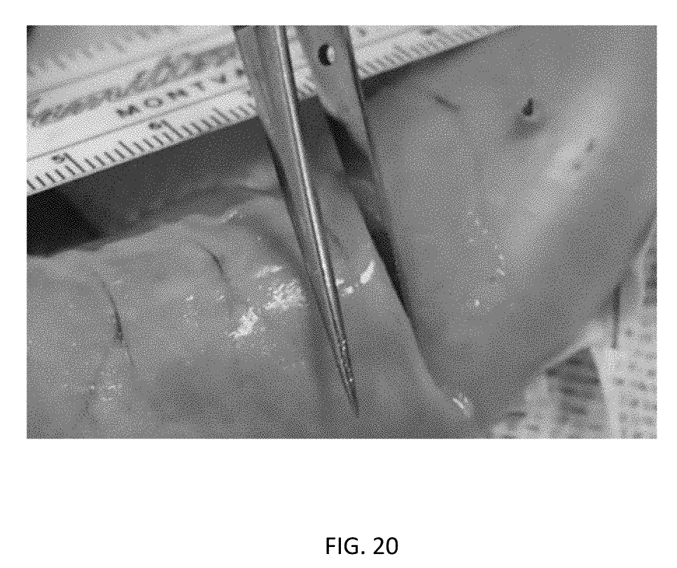

An incision over 1.5 cm in length was made in a pig colon. The material of Examples 1-3 was applied as before (see FIG. 19). The material was smoothed and solid cured as described in Examples 10 and 11. After 4 minute of cure time, the resulting film that forms could not be delaminated using forceps pressure (see FIG. 20).

### EXAMPLE 13

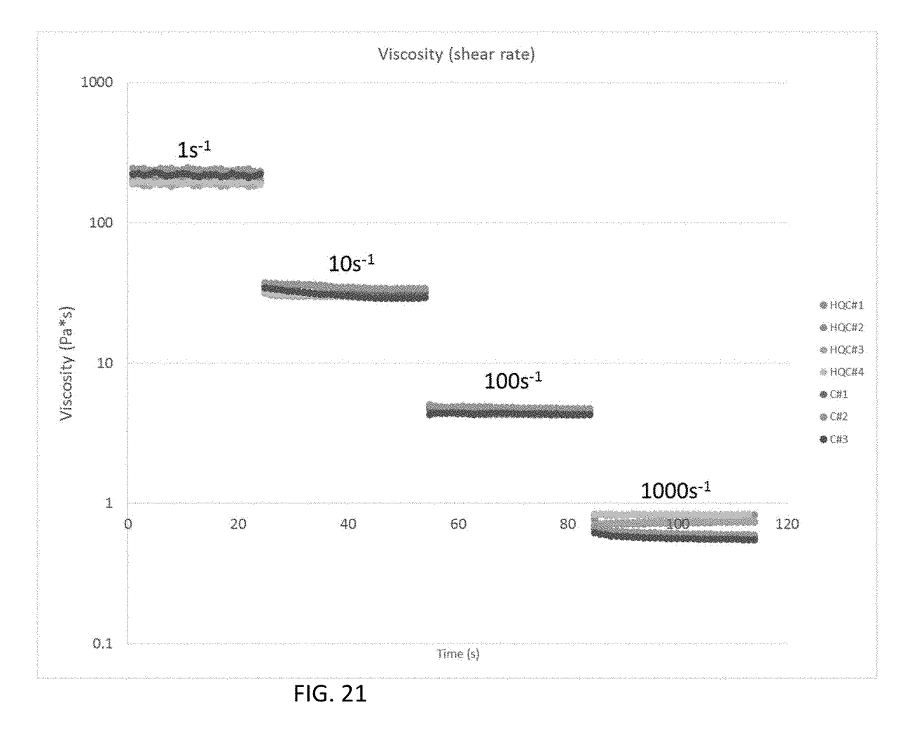

Shear rates of the porcine atelocollagen used in to produce the materials described in the above specific examples was compared to the shear rate of bovine atelocollagen. The concentration of the porcine collagen was about 38 mg/mL. The concentration of the bovine atelocollagen was about 22 mg/mL. As shown in the graphs of FIG. 21, the shear rates of the two materials were comparable even though the porcine collagen was present at a much larger concentration. These results are consistent with the porcine collagen being of a higher quality collagen (HQC), e.g., comprising more monomeric triple helices, than the bovine collagen. The shear rate tests were performed by subjecting a sample to shear stress and measuring the resulting torque expended by the viscous solution in response to this shear stress. The rheometer used in the tests was equipped with parallel plates. The lower stationary plate was heated to 25° C., and the upper rotating plate was 50 mm diameter and the distance between the plates was 1 mm. The viscosity test was performed at the following shear rates: 1 second⁻¹, 10 seconds⁻¹, 100 second⁻¹ and 1000 second⁻¹. The y-axis of FIG. 21 represents viscosity in Pascal-seconds, and the x-axis represents time in seconds. HQC (#1, #2 and #3) represent different measurements of the porcine atelocollagen, and C#1, C#2 and C#3 represent different measurements of the bovine atelocollagen.

### EXAMPLE 14

Six (6) separate experiments were conducted to examine the effect of both particle size and curing component composition on burst strength of the material tested of Examples 1-3. The following powdered curing components were tested: (a) Sodium Carbonate/Sodium Dihydrogen Phosphate, as received (CO3/PO4); (b) Sodium Carbonate/Sodium Dihydrogen Phosphate, <125 micron particle size (CO3/PO4 Fine); (c) Sodium Carbonate, as received (CO3); (d) Sodium Carbonate, <125 micron particle size (CO3 Fine); (e) Sodium Dihydrogen Phosphate, as received; and (f) Sodium Dihydrogen Phosphate, <125 micron particle size Curing components were purchased from Sigma Aldrich. Components tested “as received” were not modified in any way. Curing components marked as “fine” were ground into a finer powder using a mortar and pestle. The ground powder was passed through a series of mesh sieves. The finest powder, passing through a mesh size of 120, corresponding to a particle size of less than 125 microns, was used as the tested curing component.

Burst strength testing was conducted according to ASTM F2392-04. Briefly, Nippi sausage casing was washed overnight with 0.1M NaOH to remove glycerol residue used for preservation of the sausage casing. Casing was then rinsed three times using deionized water and stored in deionized water until ready for use. Sections of casing were cut to fit the sample testing cell. A 3 mm diameter defect was created in the center of each sausage casing sample using a surgical punch. The casing was laid flat and blotted dry with a Kimwipe™ wipe. A silicone mask (0.5 mm thickness) consisting of a circular hole measuring 15 mm in diameter was placed over the casing, centering the mask with the 3 mm defect.

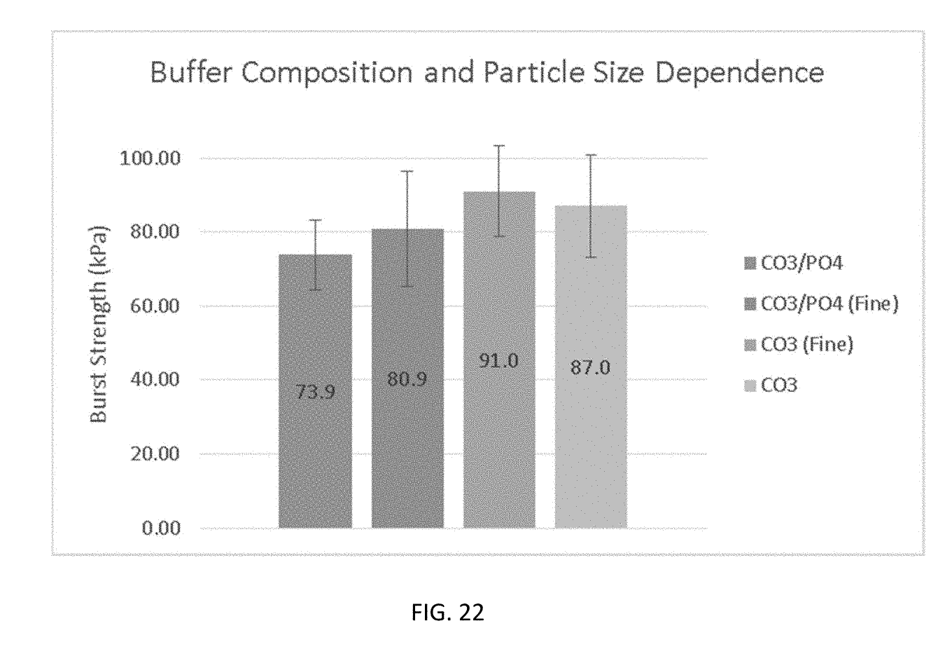

The material was prepared as described above (methylated collagen and PEG components mixed together) and approximately 0.2 mL of the material was applied to the defect. A silicone spreader was used to even distribute the applied material across the surface of the casing, assuring a uniform application and minimizing the presence of air bubbles. The material was cured for 4 minutes using the selected powdered curing component, which was applied using a powder sprayer. After 4 minutes, the sample was rinsed with deionized water to remove any residual curing component and the sample was loaded into the testing cell. Testing temperature was approximately 25° C. Samples were tested until rupture. Peak pressure at the time of rupture was recorded, as well as the mode of failure. Ten data points were collected for each curing component composition (n=10). The results are shown in FIG. 22.

The curing components comprised of solely sodium dihydrogen phosphate (both as received and fine) failed to properly cure the material within 4 minutes. After 4 minutes, the material resembled a soft boiled egg white and could not pass burst strength testing. Accordingly, no data exists for these curing components. The results of the other 4 curing components can be seen in FIG. 22.

The CO3/PO4 data represents the current powdered curing component used in the examples above. A minor improvement can be seen by using a finely ground powder of the same composition (compare 73.9 kPa to 80.9 kPa), most likely due to easier diffusion of the salt through the material. By removing the phosphate salt from the original curing component, additional improvement in burst strength can be obtained (compare 73.9 kPa to 87.0 kPa). Lastly, the greatest average burst strength is seen in the finely ground sodium carbonate powder (91.0 kPa). The results are consistent with a single carbonate powder of average particle size lower than 125 microns as providing improved burst strength.

### EXAMPLE 15

A mixture of mammalian, predominately Type I, either dermis or tendon-derived ,predominantly monomeric atelocollagen in dry form (as opposed to solution) from bovine or porcine sources is added to a solution of 0.9 M diethyl ether and 0.1 M hydrochloric acid prepared in methanol (methylation solution) at a concentration of 2% (w/w) and allowed to react for 72 hours. The collagen is generally in a granular particle form but powders, grains, pellets or other solid shapes are also suitable.

Following methylation, the methylation solution is decanted off and the methylated atelocollagen is washed and filtered three times using absolute ethanol, 10 minutes for each wash. The methylated atelocollagen is then placed under vacuum for 18 hours to remove any remaining solvent, producing dry methylated atelocollagen.

Once dried, the material is added to an aqueous buffered solution (pH 3.5) to produce a desired final collagen concentration (e.g., between 25-60 mg/mL), as required and/or desired by the specific application.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1-100. (canceled)

101. A method of repairing a tissue defect comprising:
disposing a composition at a defect site of the tissue, the composition comprising a derivatized atelocollagen comprising a number average molecular weight of less than or equal to 300 kDa, a first crosslinking agent and a second crosslinking agent different than the first crosslinking agent;
adding a curing agent to the disposed composition; and
permitting the curing agent to remain on a surface of the disposed composition for a curing period.

102. The method of claim 101, further comprising rinsing any excess curing agent from the disposed composition after the curing period.

103. The method of claim 101, further comprising adding the curing agent as a solid to the disposed composition.

104. The method of claim 101, further comprising adding the curing agent as a liquid to the disposed composition.

105. The method of claim 101, further comprising configuring the derivatized atelocollagen as methylated atelocollagen.

106. The method of claim 105, configuring one of the first and second crosslinking agents to comprise a succinimidyl group.

107. The method of claim 105, configuring one of the first and second crosslinking agents to comprise a sulfhydryl group.

108. The method of claim 101, further comprising configuring the composition to comprise an acidic pH.

109. The method of claim 101, further comprising layering the composition over the defect site prior to curing the composition.

110. The method of claim 101, further comprising configuring the derivatized atelocollagen as methylated atelocollagen, configuring one of the first and second cros slinking agents to comprise a succinimidyl group and configuring the other crosslinking agent to comprise a sulfhydryl group.

111. A method of repairing an epithelial tissue defect comprising:
disposing a composition at a defect site of the epithelial tissue, the composition comprising a derivatized atelocollagen comprising a number average molecular weight of less than or equal to 300 kDa, a first crosslinking agent and a second crosslinking agent different than the first crosslinking agent;
adding a curing agent to the disposed composition; and
permitting the added solid curing agent to remain on a surface of the disposed composition for a curing period.

112. The method of claim 111, further comprising selecting the epithelial tissue to be pulmonary tissue.

113. The method of claim 111, further comprising selecting the epithelial tissue to be blood vessel tissue.

114. The method of claim 111, further comprising selecting the epithelial tissue to be gastrointestinal tissue.

115. The method of claim 111, further comprising rinsing any excess curing agent from the disposed composition after the curing period.

116. The method of claim 111, further comprising adding the curing agent as a solid to the disposed composition.

117. The method of claim 111, further comprising adding the curing agent as a liquid to the disposed composition.

118. The method of claim 111, further comprising configuring the derivatized atelocollagen as methylated atelocollagen and configuring one of the first and second crosslinking agents to comprise a succinimidyl group.

119. The method of claim 111, further comprising configuring the derivatized atelocollagen as methylated atelocollagen and configuring one of the first and second crosslinking agents to comprise a sulfhydryl group.

120. The method of claim 111, further comprising configuring the derivatized atelocollagen as methylated atelocollagen, configuring one of the first and second cros slinking agents to comprise a succinimidyl group and configuring the other crosslinking agent to comprise a sulfhydryl group.

121-174. (canceled)
